(19) United States
(12) Patent Application Publication
(10) Pub. No.: US 2025/0160972 A1
(43) Pub. Date: May 22, 2025

(54) CONFLICT RESOLUTION FOR ACTIVATION MODE DETERMINATION OF AN ENERGY DEVICE

(71) Applicant: Cilag GmbH International, Zug (CH)

(72) Inventors: Jacqueline Corrigan Aronhalt, Loveland, OH (US); Cameron Delos McLain, Cincinnati, OH (US); Curtis Anthony Maples, Cincinnati, OH (US); Jorge Nicolas Velez Martinez, Louisville, KY (US); Stuart W. A. Hilsmier, Cincinnati, OH (US); Andrew Scott Meyers, Cincinnati, OH (US)

(21) Appl. No.: 18/810,407

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/00

(52) U.S. Cl. CPC A61B 34/25; A61B 34/76; A61B 2034/258; A61B 2560/0204

(57) ABSTRACT

Systems, methods, and instrumentalities are disclosed for dynamically determining an activation mode (e.g., energy modality and/or energy level) of a surgical instrument based on monitored data. The surgical instrument may be a combination energy device capable of delivering ultrasonic energy and radiofrequency energy. The surgical instrument may monitor data associated with a surgical procedure. The surgical instrument may select an activation mode from the plurality of activation modes based on the monitored data. The surgical instrument may deliver energy of an energy modality associated with the selected activation mode. Monitored data may include visual data, electrical data, and/or mechanical data.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
Attorney Docket No. Ser. No. 18/810,222, filed Aug. 20, 2024,
Attorney Docket No. Ser. No. 18/810,274, filed Aug. 20, 2024,
Attorney Docket No. Ser. No. 18/810,346, filed Aug. 20, 2024,
Attorney Docket No. Ser. No. 18/810,355, filed Aug. 20, 2024, and
Attorney Docket No. Ser. No. 18/810,361, filed Aug. 20, 2024.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slow to implement systems or procedures utilizing newer and improved technologies due to patient safety and a general desire for maintaining traditional practices.

In a surgical environment, smart energy devices (e.g., smart electrosurgical devices) may be incorporated. A smart energy device may be powered by a generator to effect tissue fastening, dissecting, cutting, and/or coagulation. A smart energy device may be configured for use in open surgical procedures or other procedures such as laparoscopic, endoscopic, and robotic-assisted procedures.

### SUMMARY

Operating electrosurgical instruments may require specialized training and expertise to operate safely and effectively. Surgeons may need to be familiar with the capabilities and limitations of electrosurgical instruments and their generators, as well as the specific techniques and procedures for using them in different surgical specialties. Additionally, electrosurgical instruments should be used with caution to avoid unintended tissue damage or injury to the patient. For example, the electrosurgical instrument may be configured to cut, coagulate, and/or ablate tissue during surgical procedures and an incorrect button press of an electrosurgical instrument may result in an unintended configuration.

Systems, methods, and instrumentalities are disclosed for dynamically determining an activation mode of a surgical instrument (e.g., an electrosurgical instrument) based on monitored data. An activation mode (e.g., each activation mode) may be associated with an energy modality (e.g., ultrasonic energy or radiofrequency energy) and/or an energy level. The surgical instrument may be configured to operate in a plurality of activation modes. For example, the surgical instrument may be configured to operate in a first activation mode associated with radio frequency (RF) energy and operate in a second activation mode associated with ultrasonic energy.

The surgical instrument may be configured with communication, sensing and/or feedback capabilities to monitor (e.g., receive and/or collect) data associated with a surgical procedure. Monitored data may include an internally generated data stream and/or an externally supplied data stream (e.g., data received from other surgical device(s), surgical instrument(s) and/or surgical system(s) to determine or select an appropriate activation mode (e.g., for ultrasonic and/or electrosurgical applications). Monitored data may include electrical data, mechanical data, and/or visual data. The electrical data may indicate at least one of: an impedance, a temperature, and/or a force encountered by a jaw of the surgical instrument (e.g., a tissue characteristic encountered by the jaw). The mechanical data may indicate at least one of: a jaw gap position of the jaw, a clamp force of the jaw, a stapler closure load force, and/or a cartridge selection. The visual data may be received from an advanced imaging system and may include one or more of MRI data, ultra sound data, (EBUS) data, and/or camera data.

The surgical device may use monitored data to select an activation mode. For example, the surgical instrument may be configured to determine what type of surgical procedure is being performed. The surgical instrument may select an activation mode associated with an energy modality based on, for example, an expected tissue profile for the surgical procedure. The surgical instrument may adjust the activation mode (e.g., a selected energy modality being delivered) throughout the course of the surgical procedure (e.g., rather than just on a procedure-by-procedure basis).

Dynamic determination of an activation mode of the surgical instrument based on monitored data may enable simplification of user interaction with the surgical instrument. Delivery of an energy associated with an activation mode (e.g., energy modality) may be based on actuation of a single input source (e.g., a button) of the surgical instrument. Limiting user input to a single input source may expand functionality of the surgical instrument while minimizing user interface complexity.

The surgical instrument may be configured to determine an actuation event associated with a button of the surgical instrument. The actuation event may be based on one or more of: an actuation duration, a number of actuations, and/or an actuation force. The surgical instrument may select an activation mode based on one or more of: detected actuation events, time between actuation events, and/or sequences of actuation events (e.g., in the context of a determined surgical procedure).

In examples, the surgical instrument may have a default activation mode. For example, if actuation is not detected for a threshold period of time, the default activation mode may be selected. In examples, the surgical instrument may maintain an activation mode. For example, if actuation occurs frequently, the surgical instrument may maintain an activation mode. For example if time between actuations is below a threshold period of time, the previously selected activation mode may be selected.

The surgical instrument may provide an indication of the selected activation mode. The indication of the selected activation mode may include one or more of: haptic feedback, a visual indicator, and/or an audible indicator.

Operating electrosurgical instruments may require specialized training and expertise to operate safely and effectively. Surgeons may need to be familiar with the capabilities and limitations of electrosurgical instruments and their generators, as well as the specific techniques and procedures for using them in different surgical specialties. Additionally, electrosurgical instruments should be used with caution to avoid unintended tissue damage or injury to the patient. For example, the electrosurgical instrument may be configured to cut, coagulate, and/or ablate tissue during surgical procedures and an incorrect button press of an electrosurgical instrument may result in an unintended configuration.

Dynamic determination of an activation mode of the surgical instrument based on monitored data may enable simplification of user interaction with the surgical instrument. Delivery of an energy associated with an activation mode (e.g., energy modality and/or energy level) may be based on actuation of a single input source (e.g., a button) of the surgical instrument. Limiting user input to a single input source may expand functionality of the surgical instrument while minimizing user interface complexity.

Systems, methods, and instrumentalities are disclosed for dynamically determining an activation mode of a surgical instrument (e.g., an electrosurgical instrument) based on monitored data. The surgical instrument may be configured to operate in multiple activation modes. An activation mode (e.g., each activation mode) may be associated with an energy modality (e.g., ultrasonic energy or radiofrequency energy). For example, the surgical instrument may be configured to operate in a first activation mode associated with radio frequency (RF) energy and operate in a second activation mode associated with ultrasonic energy.

The surgical instrument may be configured with communication, sensing and/or feedback capabilities to monitor (e.g., receive and/or collect) data associated with a surgical procedure. Monitored data may include an internally generated data stream and/or an externally supplied data stream (e.g., data received from other surgical instrument(s), device(s), and/or system(s)) to determine or select an appropriate activation mode (e.g., for ultrasonic and/or electrosurgical applications). Monitored data may include visual data. The visual data may be received from an advanced imaging system and may include MRI data, ultra sound data, (EBUS) data, camera data and/or the like.

The surgical device may use monitored data (e.g., visual data) to select an activation mode. For example, the surgical instrument may be configured to determine what type of surgical procedure is being performed. The surgical instrument may select an activation mode associated with an energy modality and/or an energy level based on, for example, an expected tissue profile for the surgical procedure. The surgical instrument may adjust the activation mode (e.g., a selected energy modality) throughout the course of the surgical procedure (e.g., rather than just on a procedure-by-procedure basis).

The visual data may be associated with a surgical site. The surgical instrument may determine, based on the visual data, a tissue characteristic of a tissue in the surgical site. The tissue characteristic may include a tissue thickness, an organ associated with the tissue, a tissue composition, a tissue location, a tissue tension, and/or a tissue deformation. The surgical instrument may select an activation mode based on the tissue characteristic. For example, a first activation mode (e.g., associated with RF energy) may be selected if the determined tissue tension is below a threshold value, and the second activation mode (e.g., an activation mode associated with ultrasonic energy) may be selected if the tissue tension is above the threshold value.

The surgical instrument may determine, based on the visual data, an instrument characteristic (e.g., of the surgical instrument). The instrument characteristic may include a location of the surgical instrument within the surgical site, an orientation of the surgical instrument, a current surgical task, and/or or proximity to a second surgical instrument. The surgical instrument may select an activation mode based on the instrument characteristic.

The surgical instrument may monitor visual data from multiple sources. For example, the surgical instrument may obtain first visual data (e.g., camera data) from a first imaging system and obtain second visual data (e.g., MRI data) from a second imaging system. The surgical instrument may include an imaging system (e.g., a camera) that may be used to collect visual data.

Operating electrosurgical instruments may require specialized training and expertise to operate safely and effectively. Surgeons may need to be familiar with the capabilities and limitations of electrosurgical instruments and their generators, as well as the specific techniques and procedures for using them in different surgical specialties. Additionally, electrosurgical instruments should be used with caution to avoid unintended tissue damage or injury to the patient. For example, the electrosurgical instrument may be configured to cut, coagulate, and/or ablate tissue during surgical procedures and an incorrect button press of an electrosurgical instrument may result in an unintended configuration.

Dynamic determination of an activation mode of the surgical instrument based on monitored data may enable simplification of user interaction with the surgical instrument. Delivery of an energy associated with an activation mode (e.g., energy modality) may be based on actuation of a single input source (e.g., a button) of the surgical instrument. Limiting user input to a single input source may expand functionality of the surgical instrument while minimizing user interface complexity.

Systems, methods, and instrumentalities are disclosed for dynamically determining an activation mode of a surgical instrument (e.g., an electrosurgical instrument) based on monitored data. The surgical instrument may be configured to operate in multiple activation modes. An activation mode (e.g., each activation mode) may be associated with an energy modality (e.g., ultrasonic energy or radiofrequency energy). For example, the surgical instrument may be configured to operate in a first activation mode associated with radio frequency (RF) energy and operate in a second activation mode associated with ultrasonic energy.

The surgical instrument may be configured with communication, sensing and/or feedback capabilities to monitor (e.g., receive and/or collect) data associated with a surgical procedure. Monitored data may include an internally generated data stream and/or an externally supplied data stream (e.g., data received from other surgical instrument(s), device(s), and/or system(s)) to determine or select an appropriate activation mode (e.g., for ultrasonic and/or electrosurgical applications). Monitored data may include electrical data. The electrical data may indicate one or more of: an impedance, a temperature, and/or a force encountered by a jaw of the surgical instrument (e.g., a tissue characteristic encountered by the jaw).

The surgical device may use monitored electrical data to select an activation mode. For example, the surgical instrument may be configured to determine what type of surgical procedure is being performed. The surgical instrument may select an activation mode associated with an energy modality based on, for example, an expected tissue profile for the surgical procedure. The surgical instrument may adjust the activation mode (e.g., a selected energy modality) throughout the course of the surgical procedure (e.g., rather than just on a procedure-by-procedure basis). For example, if monitored electrical data indicates a transaction being performed is complete, an activation mode associated with ultrasonic energy may be selected. In examples, a use time of the surgical instrument may be used to selected an activation mode.

The electrical data may be associated with a surgical site. The surgical instrument may determine, based on the electrical data, a tissue characteristic of a tissue in the surgical site. For example, the electrical data may indicated one or more of: an impedance, a temperature, or a force (e.g., encountered by a jaw of the surgical instrument when in contact with the tissue). The tissue characteristic may be or may include, for example, a tissue thickness, an organ associated with the tissue, and/or a tissue composition. The surgical instrument may select an activation mode based on the tissue characteristic.

In examples, the surgical instrument may determine, based on the electrical data, an impedance associated with a tissue in the surgical site. If the impedance associated with the tissue is above a threshold value, a first activation mode (e.g., an activation mode associated with RF energy) may be selected, and if the impedance associated with the tissue is below the threshold value, a second activation mode (e.g., an activation mode associated with ultrasonic energy) may be selected.

In examples, the surgical instrument may determine, based on the monitored electrical data, a density associated with a tissue in the surgical site. The surgical instrument may determine a tissue type based on the determined density. The surgical instrument may select an activation mode based on the determined tissue type and/or determined density.

The surgical instrument may monitor electrical data following a sub-therapeutic pulse (e.g., to assess the nearby tissue conditions and tissue status). Electrical data may be used in conjunction with (e.g., compared to) other types of data (e.g., visual data, mechanical data) to verify determined tissue characteristics and/or to select an activation mode.

Operating electrosurgical instruments may require specialized training and expertise to operate safely and effectively. Surgeons may need to be familiar with the capabilities and limitations of electrosurgical instruments and their generators, as well as the specific techniques and procedures for using them in different surgical specialties. Additionally, electrosurgical instruments should be used with caution to avoid unintended tissue damage or injury to the patient. For example, the electrosurgical instrument may be configured to cut, coagulate, and/or ablate tissue during surgical procedures and an incorrect button press of an electrosurgical instrument may result in an unintended configuration.

Dynamic determination of an activation mode of the surgical instrument based on monitored data may enable simplification of user interaction with the surgical instrument. Delivery of an energy associated with an activation mode (e.g., energy modality and/or energy level) may be based on actuation of a single input source (e.g., a button) of the surgical instrument. Limiting user input to a single input source may expand functionality of the surgical instrument while minimizing user interface complexity.

Systems, methods, and instrumentalities are disclosed for dynamically determining an activation mode of a surgical instrument (e.g., an electrosurgical instrument) based on monitored data. The surgical instrument may be configured to operate in multiple activation modes An activation mode (e.g., each activation mode) may be associated with an energy modality (e.g., ultrasonic energy or radiofrequency energy) and/or an energy level. For example, the surgical instrument may be configured to operate in a first activation mode associated with radio frequency (RF) energy and operate in a second activation mode associated with ultrasonic energy.

The surgical instrument may be configured with communication, sensing, and/or feedback capabilities to monitor (e.g., receive and/or collect) data associated with a surgical procedure. Monitored data may include an internally generated data stream and/or an externally supplied data stream (e.g., data received from other surgical instrument(s), device(s), and/or system(s)) to determine or select an appropriate activation mode (e.g., for ultrasonic and/or electrosurgical applications). Monitored data may include mechanical data, which may indicate at least one of: a jaw gap position of the jaw, a clamp force of the jaw, a stapler closure load force, and/or a cartridge selection.

The surgical device may use monitored mechanical data to select an activation mode. For example, the surgical instrument may be configured to determine what type of surgical procedure is being performed. The surgical instrument may select an activation mode associated with an energy modality based on, for example, an expected tissue profile for the surgical procedure. The surgical instrument may adjust the activation mode (e.g., a selected energy modality) throughout the course of the surgical procedure (e.g., rather than just on a procedure-by-procedure basis). For example, if the mechanical data indicates a current surgical task is being performed that includes feathering or tissue marching a particular activation mode (e.g., an activation mode associated with ultrasonic energy) may be selected.

Monitored mechanical data may be associated with the surgical instrument. The mechanical data may include a jaw gap position indication (e.g., fully open, partially closed, fully closed) associated with a jaw of the surgical instrument; a tissue presence indication (e.g., associated with a jaw of the surgical instrument), and/or a jaw force indication (e.g., a detected jaw force, detected load). The surgical instrument may select an activation mode based on the jaw gap position indication, the tissue presence indication, and/or the jaw force indication.

For example, if the jaw gap position indication indicates that the jaw of the surgical instrument is closed and the tissue presence indication indicates a tissue is present, a particular activation mode (e.g., an activation mode associated with RF energy) may be selected.

For example, if the jaw gap position indication indicates that the jaw of the surgical instrument is closed and the tissue presence indication indicates no tissue is present, a particular activation mode (e.g., an activation mode associated with ultrasonic energy) may be selected.

For example, if the jaw force indication indicates a detected load is less than a threshold load and the jaw gap indication indicates the jaw is open, a particular activation mode (e.g., an activation mode associated with ultrasonic energy) may be selected.

The surgical instrument may determine, based on mechanical data, a blade deflection indication (e.g., a distance between a blade of the surgical instrument and an inner tube of the surgical instrument). The blade deflection indication may be determined by an emitter of the surgical instrument and/or visual data. The surgical instrument may select an activation mode based on the blade deflection indication. The surgical instrument may provide a warning if the blade deflection indication indicates blade deflection is above a threshold (e.g., repositioning of the surgical instrument and/or a device assessment is required).

Based on the mechanical data, the surgical instrument may be configured to determine an actuation event associated with a button of the surgical instrument. The actuation event may be based on one or more of: an actuation duration, a number of actuations, and/or an actuation force. The surgical instrument may select an activation mode based on one or more of: detected actuation events, time between actuation events, and/or sequences of actuation events (e.g., in the context of a determined surgical procedure).

Operating electrosurgical instruments may require specialized training and expertise to operate safely and effectively. Surgeons may need to be familiar with the capabilities and limitations of electrosurgical instruments and their generators, as well as the specific techniques and procedures for using them in different surgical specialties. Additionally, electrosurgical instruments should be used with caution to avoid unintended tissue damage or injury to the patient. For example, the electrosurgical instrument may be configured to cut, coagulate, and/or ablate tissue during surgical procedures and an incorrect button press of an electrosurgical instrument may result in an unintended configuration.

Dynamic determination of an activation mode of the surgical instrument based on monitored data may enable simplification of user interaction with the surgical instrument. Delivery of an energy associated with an activation mode (e.g., energy modality and/or energy level) may be based on actuation of a single input source (e.g., a button) of the surgical instrument. Limiting user input to a single input source may expand functionality of the surgical instrument while minimizing user interface complexity.

Systems, methods, and instrumentalities are disclosed for dynamically determining an activation mode of a surgical instrument (e.g., an electrosurgical instrument) based on monitored data, including a combination of electrical data, mechanical data, and/or visual data. An activation mode (e.g., each activation mode) may be associated with an energy modality (e.g., ultrasonic energy or radiofrequency energy). The surgical instrument may be configured to operate in multiple activation modes. For example, the surgical instrument may be configured to operate in a first activation mode associated with radio frequency (RF) energy and operate in a second activation mode associated with ultrasonic energy.

The surgical instrument may be configured with communicating, sensing, and/or feedback capabilities to monitor (e.g., receive and/or collect) data associated with a surgical procedure. Monitored data may include an internally generated data stream and/or an externally supplied data stream (e.g., data received from other surgical instrument(s), device(s) and/or system(s)) to determine or select an appropriate activation mode (e.g., for ultrasonic and/or electrosurgical applications). Monitored data may include electrical data, mechanical data, and/or visual data. The electrical data may indicate at least one of: an impedance, a temperature, and/or a force encountered by a jaw of the surgical instrument (e.g., a tissue characteristic encountered by the jaw). The mechanical data may indicate at least one of: a jaw gap position of the jaw, a clamp force of the jaw, a stapler closure load force, and/or a cartridge selection. The visual data may be received from an imaging system and may include at least one of: MRI data, ultra sound data, (EBUS) data, or camera data.

The surgical device may use monitored data to select an activation mode. The monitored data may be associated with a surgical site. For example, the surgical instrument may be configured to determine what type of surgical procedure is being performed. The surgical instrument may select an activation mode associated with an energy modality based on, for example, an expected tissue profile for the surgical procedure. The surgical instrument may adjust the activation mode (e.g., a selected energy modality) throughout the course of the surgical procedure (e.g., rather than just on a procedure-by-procedure basis).

The surgical instrument may determine (e.g., based on the visual data and/or electrical data) a tissue characteristic of a tissue in the surgical site. The tissue characteristic may include a tissue thickness, an organ associated with the tissue, a tissue composition, a tissue location, a tissue tension, and/or a tissue deformation. The surgical instrument may assess the nearby tissue conditions and tissue status based on data monitored following a sub-therapeutic pulse. The surgical instrument may select an activation mode based on the tissue characteristic. For example, a first activation mode (e.g., associated with RF energy) may be selected if the determined tissue tension is below a threshold value and the second activation mode (e.g., an activation mode associated with ultrasonic energy) may be selected if the tissue tension is above the threshold value.

The surgical instrument may determine (e.g., based on the visual data and/or mechanical data) an instrument characteristic (e.g., of the surgical instrument). The instrument characteristic may include one or more of: a location of the surgical instrument within the surgical site, an orientation of the surgical instrument; a current surgical task, or proximity to a second surgical instrument The surgical instrument may select an activation mode based on the instrument characteristic.

The surgical instrument may determine (e.g., based on visual data and mechanical data) a blade deflection indication (e.g., a distance between a blade of the surgical instrument and an inner tube of the surgical instrument). The blade deflection indication may be determined by an emitter of the surgical instrument and/or visual data. The surgical instrument may select an activation mode based on the blade deflection indication. The surgical instrument may provide a warning if the blade deflection indication indicates blade deflection is above a threshold (e.g., repositioning of the surgical instrument and/or a device assessment is required).

The surgical instrument may monitor data (e.g., visual data, electrical data, and/or mechanical data) from multiple sources. Monitored data (e.g., data from different sources) may be analyzed to determine if the data reflects consistent information. Consideration and/or weighting (e.g., by the surgical instrument) of indications from monitored data may depend on a determined consistency of the data. For example, the surgical instrument may determine whether a predicted activation mode is consistent with monitored data across sources. If a determination is made that the predicted activation mode is consistent (e.g., across types of data and/or data sources), the predicted activation mode may be selected by the surgical instrument.

For example, the surgical instrument may obtain visual data (e.g., camera data) from an imaging system that indicates a tissue characteristic and may collect electrical data from a sensor of the surgical instrument. The surgical instrument may determine whether the electrical data from the surgical instrument confirms the tissue characteristic (e.g., as indicated by the visual data). If the electrical data from the surgical instrument confirms the tissue characteristic, a particular activation mode may be selected based on the tissue characteristic. If the electrical data from the surgical instrument does not confirm the tissue characteristic, the surgical instrument may not consider, or consider to a lesser degree, the tissue characteristic when selecting an activation mode.

For example, the surgical instrument may obtain mechanical data from the surgical instrument and the visual data (e.g., from an imaging system). The surgical instrument may determine the mechanical data indicates a sequence of rapid jaw closures of a jaw of the surgical instrument. The surgical instrument may determine (e.g., based on the visual data and/or a jaw position indication) a tissue thickness of a tissue within a jaw of the surgical instrument. Based on determining the sequence of rapid jaw closures and the determined tissue thickness is above a thickness threshold, a particular activation mode (e.g., an activation mode associated with ultrasonic energy) may be selected.

For example, the surgical instrument may obtain mechanical data from the surgical instrument and the visual data (e.g., from an imaging system). The surgical instrument may determine the mechanical data indicates a jaw force of a jaw of the surgical instrument and, based on the visual data from the imaging system, that blade deflection of a blade of the surgical instrument has occurred. Based on the determined jaw force and determining that blade deflection has occurred, the surgical instrument may select a particular activation mode (e.g., an activation mode associated with ultrasonic energy).

For example, the surgical instrument may obtain mechanical data from the surgical instrument and the electrical data. The surgical instrument may determine (e.g., based on the mechanical data) a jaw of the surgical instrument is closed. The surgical instrument may determine an impedance of a tissue located in the jaw of the surgical instrument (e.g., based on the electrical data). Based on the determined impedance of the tissue being above a threshold impedance, and that the jaw is closed, the surgical instrument may select a particular activation mode (e.g., an activation mode associated with RF energy.

A surgical instrument may monitor data (e.g., visual data, electrical data, mechanical data) from multiple sources. Monitored data (e.g., data from different sources) may be analyzed to determine if it is consistent. Consideration and/or weighting (e.g., by the surgical instrument) of indications from monitored data may depend on a determined consistency of the data (e.g., whether conflicting analysis is present). For example, the surgical instrument may determine whether a predicted activation mode is consistent with monitored data across sources. If a determination is made that the predicted activation mode is consistent (e.g., across types of data and/or data sources), the predicted activation mode may be selected by the surgical instrument. If a determination is made that the predicted activation mode is inconsistent, the surgical instrument may perform conflict resolution (e.g., if monitored data is inconsistent, corrupted, or outside of an expected range). For example, the surgical instrument may be configured to self-disable specific activation modes (e.g., activation mode(s) that may be associated with an energy modality) to optimize patient treatment and/or minimize risk.

For example, if data inconsistencies are observed the surgical instrument may not activate. The surgical instrument may require user mode selection (e.g., manual selection of an activation mode) if there is a gap in monitored data or data inconsistencies.

For example, the surgical instrument may determine a proximity of the surgical instrument to a biological structure within a surgical site and/or a surgical device. The surgical instrument may, based on the proximity, disable particular activation mode(s).

The surgical instrument may provide a user (e.g., a surgeon) with feedback of a selected activation mode. The surgical instrument may indicate the selected activation mode via one or more of: an audible indication, a visual indication, or haptic feedback. The surgical instrument may be configured to receive user feedback to confirm whether the user intends to use the selected activation mode (e.g., if an activation mode was selected). If a user feedback indication is a confirmation of the selected activation mode, the surgical instrument may deliver energy of the energy modality and/or energy level associated with the selected activation mode.

The surgical instrument may provide an indication of the selected activation mode. The indication of the selected activation mode may comprise one or more of: haptic feedback, a visual indicator, or an audible indicator.

### BRIEF DESCRIPTION OF THE DRAWINGS

FIG. 1 is a block diagram of a computer-implemented surgical system.

FIG. 2 depicts an example surgical system in a surgical operating room.

FIG. 3 depicts an example surgical hub paired with various systems.

FIG. 4 depicts an example situationally aware surgical system.

FIG. 5 depicts an example surgical instrument.

FIG. 6 depicts an example handle assembly of the surgical instrument shown in FIG. 5.

FIG. 7 depicts an example end effector of the surgical instrument shown in FIG. 5.

FIG. 8 depicts an example method for activation mode determination.

FIG. 9 depicts an example method of activation mode selection based on monitored visual data.

FIG. 10 depicts an example method of activation mode selection based on monitored electrical data.

FIG. 11 depicts an example method of activation mode selection based on monitored mechanical data.

FIG. 12 depicts an example method of activation mode selection based on monitored data sources.

FIG. 13 depicts an example method of selecting an activation mode with conflict resolution of data sources.

### DETAILED DESCRIPTION

A more detailed understanding may be had from the following description, given by way of example in conjunction with the accompanying drawings.

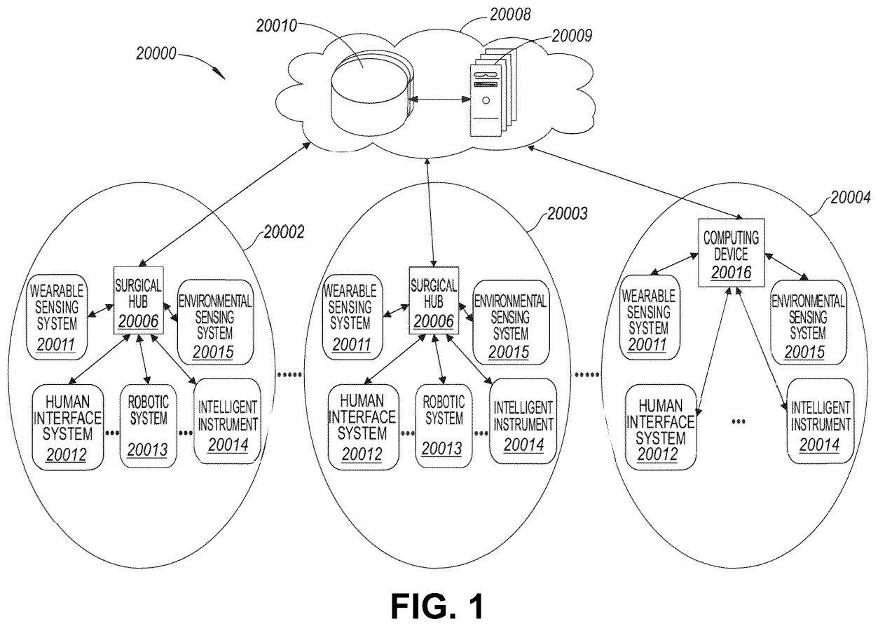

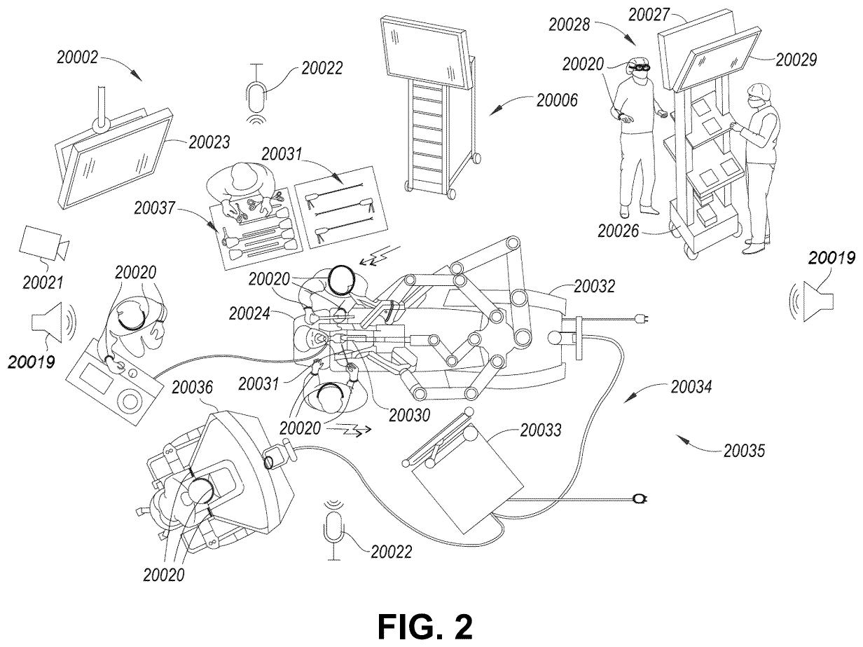

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It may be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is incorporated herein by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiogramactroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is incorporated herein by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 10013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is incorporated herein by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is incorporated herein by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is incorporated herein by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye may respond to wavelengths in air that range from about 380 nm to about 750 nm.

The invisible spectrum (e.g., the non-luminous spectrum) is the portion of the electromagnetic spectrum that lies below and above the visible spectrum (e.g., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is incorporated herein by reference in its entirety. Multi-spectrum monitoring can be a useful tool in relocating a surgical field after a current surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It may be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

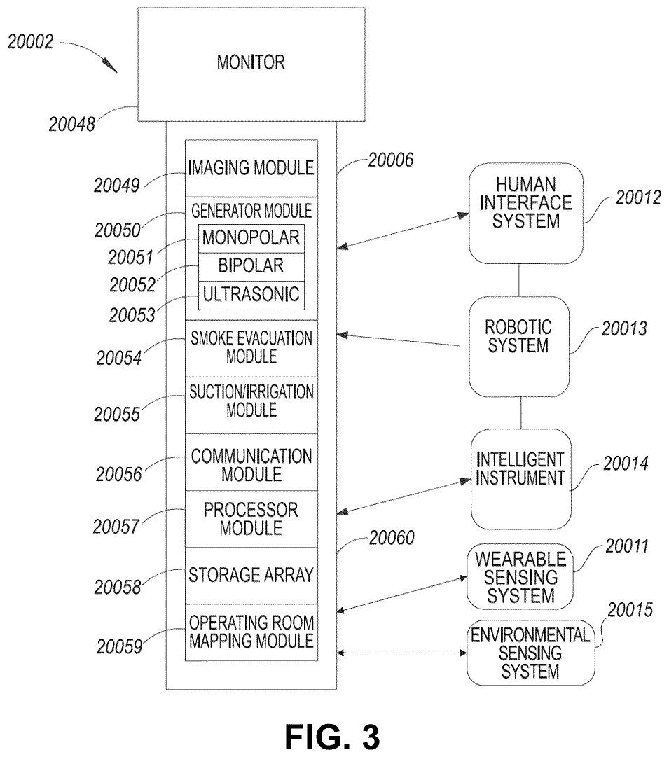

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050, a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is incorporated herein by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

Multiple energy types (e.g., energy modalities) may be applied to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

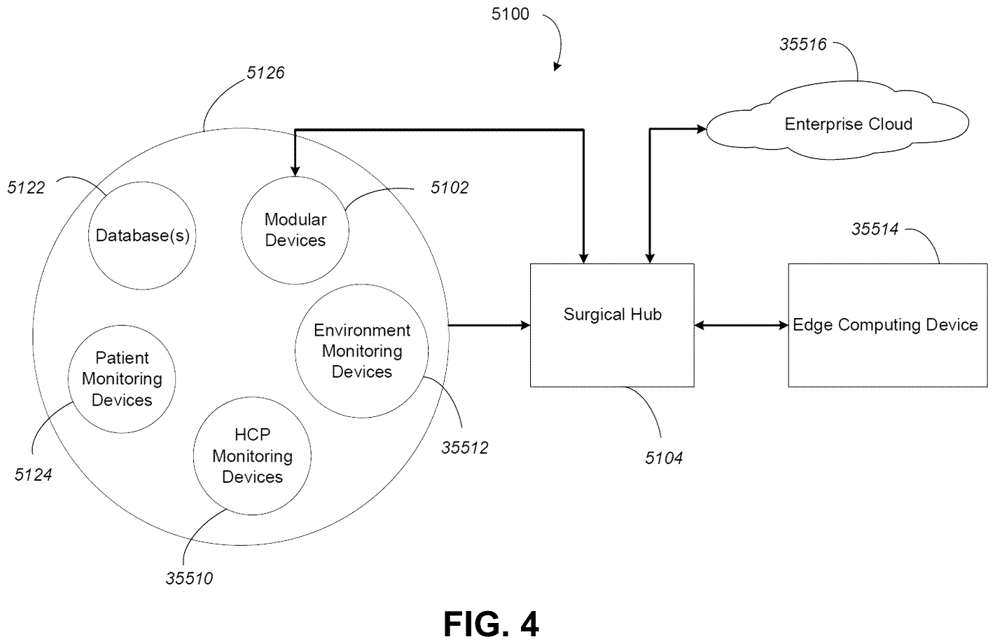

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiogramonitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient moni-toring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or may subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visual data. The additional context can be useful when the visual data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

Surgical instruments (e.g., intelligent instrument 20014) may include smart power systems. In some examples, smart power systems may be independent and may not have a fixed support point. Smart powered systems may be held and manipulated by a surgeon or health care personnel (e.g., directly). For example, smart powered systems may include one or more of: a handheld powered stapler with powered movement and/or placement (e.g., articulation) aspects; a handheld powered stapler with powered movement and/or placement (e.g., articulation and shaft rotation) aspects; or a handheld ultrasonic advanced energy device. The smart powered system may be configured to communicate to other smart systems via wired and/or wireless technologies as described herein (e.g., via Bluetooth).

Intelligent instrument 20014 may be a combination electrosurgical device (e.g., configured to deliver ultrasonic energy and RF energy) that may be handheld or tethered. For example, intelligent instrument 20014 may include an ultrasonic tissue welding and cutting system. The ultrasonic tissue welding and cutting system may be powered by a modular battery. A battery pack and/or control electronics of the ultrasonic tissue welding and cutting system may be part of a first modular portion and an ultrasonic transducer may be part of a second modular portion. The handheld system may accommodate a replaceable wave guide and/or blade that may be disposable. The modularity of a handheld system may be expanded to one or more of radio frequency (RF) monopolar, RF bipolar, and/or combination devices. The energy modalities may be blended, alternated, and/or combined based on one or more of tissue properties, jaw gap, or force. Operational parameters, such as one or more of tissue properties, jaw gap, or force may be communicated via wireless communication to other smart systems or recorded (e.g., stored).

Tethered but independent smart systems (e.g., intelligent instrument 20014) may include handheld system(s), which a surgeon or healthcare personnel may handle (e.g., orient). The handheld system(s) may be connected (e.g., by a wired tether) to a fixed piece of control electronics. A tethered but independent smart system may include an advanced energy generator for adaptive control of one or more of monopolar RF, bipolar RF, or ultrasonic tissue welding.

A tethered but independent system may include one or more of an ultrasonic generator, a RF monopolar generator, or a RF bipolar generator that may be used to control the supply of energy to an attached surgical instrument (e.g., to control the energy modality). The energy magnitude and energy modality (e.g., activation mode) may be determined, selected, and/or controlled by the surgical instrument, generator, and/or surgical hub (e.g., by sensing aspects of the tissue and/or the device). Sensing aspects are described, for example, in U.S. Pat. No. 10,842,523 (U.S. patent application Ser. No. 15/382,515), titled MODULAR BATTERY POWERED HANDHELD SURGICAL INSTRUMENT AND METHODS THEREFOR, filed Dec. 16, 2016; U.S. Pat. No. 11,051,873 (U.S. patent application Ser. No. 15/177,449), titled SURGICAL SYSTEM WITH USER ADAPTABLE TECHNIQUES EMPLOYING MULTIPLE ENERGY MODALITIES BASED ON TISSUE PARAMETERS, filed Jun. 9, 2016; and U.S. Pat. No. 10,765,470 (U.S. patent application Ser. No. 15/177,466), titled SURGICAL SYSTEM WITH USER ADAPTABLE TECHNIQUES EMPLOYING SIMULTANEOUS ENERGY MODALITIES BASED ON TISSUE PARAMETERS, filed Jun. 9, 2016, the disclosures of which are incorporated herein by reference in their entirety.

Aspects of the device and/or tissue (e.g., max applied temperature) may be used to limit the input energy. Some examples of tethered but independent smart systems and/or their capabilities are described in U.S. Pat. No. 11,589,888 (U.S. patent application Ser. No. 16/209,453), titled METHOD FOR CONTROLING SMART ENERGY DEVICES, filed Dec. 4, 2018; U.S. Patent Application Publication No. 2019/0201136 (U.S. patent application Ser. No. 16/209,395), titled METHOD OF HUB COMMUNICATION, filed Dec. 4, 2018; and U.S. Patent Application Publication No. 2019/0201112 (U.S. patent application Ser. No. 15/940,629), titled COMPUTER IMPLEMENTED INTERACTIVE SURGICAL SYSTEMS, filed Mar. 29, 2018, the disclosures of which are incorporated herein by reference in their entirety.

For example, a generator may be configured to deliver multiple energy modalities to a surgical instrument. The generator may provide RF and ultrasonic signals for delivering energy to a surgical instrument either independently or simultaneously. The RF and ultrasonic signals may be provided alone or in combination. The RF and ultrasonic signals may be provided simultaneously. The generator output can deliver multiple energy modalities (e.g., ultrasonic, bipolar or monopolar RF, irreversible and/or reversible electroporation, and/or microwave energy, among others) through a single port, and these signals can be delivered separately or simultaneously to the end effector to treat tissue. The generator may include a processor coupled to a waveform generator. The processor and waveform generator may generate a variety of signal waveforms based on information stored in a memory coupled to the processor. The digital information associated with a waveform may be provided to the waveform generator, which may include one or more DAC circuits to convert the digital input into an analog output. The analog output may be fed to an amplifier for signal conditioning and amplification. The conditioned and amplified output of the amplifier may be coupled to a power transformer. The signals may be coupled across the power transformer to the secondary side, which may be in the patient isolation side. A first signal of a first energy modality may be provided to the surgical instrument via a first energy terminal. A second signal of a second energy modality may be coupled across a capacitor and may be provided to the surgical instrument via a second energy terminal. It may be appreciated that more than two energy modalities may be output and thus the subscript “n” may be used to designate that up to n ENERGYn terminals may be provided, where n is a positive integer greater than 1. It also may be appreciated that up to “n” return paths RETURNn may be provided.

The term surgical instrument as used herein may refer to a handheld intelligent surgical instrument, such as an intelligent instrument 20014 as shown in FIGS. 1 and 3. The surgical instrument may be a combination electrosurgical device configured to deliver ultrasonic energy or radiofrequency (RF) energy (e.g., at varying power levels). A surgical system, such as surgical system 20002, and/or any of its associated surgical sub-systems, such as intelligent instrument 20014 may be configured to dynamically select an activation mode based on data monitored by surgical system 20000 (e.g., as described herein).

An activation mode (e.g., each activation mode) may be associated with an energy modality (e.g., ultrasonic energy or radiofrequency energy) and/or an energy level. A surgical instrument may be configured to operate in a plurality of activation modes. For example, the surgical instrument may be configured to operate in a first activation mode associated with radio frequency (RF) energy and operate in a second activation mode associated with ultrasonic energy. Activation modes may be configured for an intended use of the surgical instrument (e.g., whether the cut, coagulate, and/or ablate tissue during surgical procedures).

The surgical instrument (e.g., intelligent instrument 20014) may be configured with sensing and feedback capabilities to monitor (e.g., receive and/or collect) data associated with a surgical procedure. Monitored data may include an internally generated data stream and/or an externally supplied data stream (e.g., data received from other surgical systems 2002, 2003, 2004, surgical subsystems, such as environment sensing system 20015, and/or other surgical devices) to determine or select an appropriate activation mode (e.g., for ultrasonic and/or electrosurgical applications). Monitored data may comprise one or more of: electrical data, mechanical data, or visual data.

A surgical instrument may use monitored data to select an activation mode. For example, the surgical instrument may be configured to determine a type of surgical procedure being performed. The surgical instrument may select an activation mode associated with an energy modality and/or an energy level based on, for example, an expected tissue profile for the surgical procedure. The surgical instrument may adjust the activation mode (e.g., a selected energy modality and/or energy level being delivered) throughout the course of the surgical procedure (e.g., rather than just on a procedure-by-procedure basis).

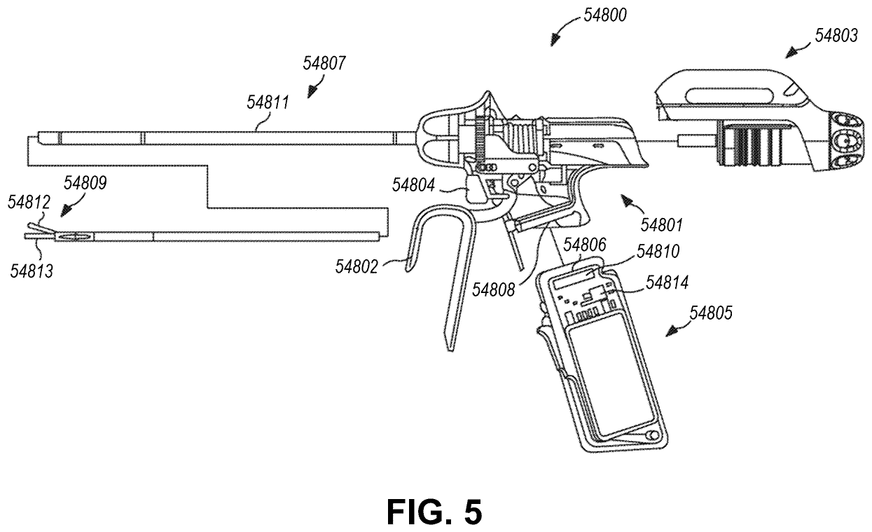

FIG. 5 depicts an elevational exploded view of an example surgical instrument 54800. Surgical instrument 54800 may be an intelligent instrument 20014 (e.g., surgical instrument 54800 may be configured to operate and/or communicate as part of a surgical system 20002, 20003, 20004). FIG. 5 depicts surgical instrument 54800 with the left shell half removed from a handle assembly 54801. Surgical instrument 54800 may comprise one or more of: handle assembly 54801, transducer/generator assembly 54803, smart battery assembly 54805, waveguide shaft assembly 54807, or end effector 54809.

Surgical instrument 54800 may be used with a variety of devices (e.g., generators), which may or may not have power requirements (e.g., current and voltage) and/or delivery capabilities that vary from one another. Surgical instrument 54800 may have a wide frequency range and output power to deliver different energy modalities (e.g., ultrasonic energy and RF energy). The lower voltage, higher current demand of electrosurgical devices may be met by a dedicated tap on a wideband power transformer, thereby eliminating the need for a separate power amplifier and output transformer. Moreover, sensing and feedback circuits of surgical instrument 54800 may support a large dynamic range (e.g., that addresses the needs of both ultrasonic and electrosurgical applications with minimal distortion).

Surgical instrument 54800 may be configured to identify a particular device to which it is electrically coupled, such as a generator (e.g., a particular model of generator). Surgical instrument 54800 may identify the device through encrypted or unencrypted identification methods. For example, surgical instrument 54800 may have connection portion 54806 (e.g., to connect to smart battery assembly 54805). The handle assembly 54801 may include a device identifier communicatively coupled to the multi-lead handle terminal assembly 54808. Surgical instrument 54800 may be configured to communicate at least one piece of information about the handle assembly 54801. This information may pertain to one or more of: a type or an identity of the generator (e.g., that may be presently connected to the handle assembly 54801), a number of times the handle assembly 54801 has been used; information regarding available activation mode(s) (e.g., energy modalities, power levels, actuation sequences associated with an activation mode, for example each activation mode); a currently selected activation mode; a last used energy modality, a time since a most recent use and/or actuation; monitored data (e.g., electrical data, mechanical data, and/or visual data); and/or other characteristics.

In examples, a communication portion of surgical instrument 54800 may include a processor 54810 and a memory 54814, (e.g., which may be separate or a single component). The processor 54810 may able to determine an appropriate energy modality (e.g., ultrasonic or RF energy) for the surgical instrument 54800 to deliver. In examples, surgical instrument 54800 may send monitored data and/or information regarding surgical instrument 54800 to a communicatively coupled generator or surgical hub. The generator or surgical hub may be configured to determine an activation mode (e.g., energy modality and/or power level) for surgical instrument 54800. Surgical instrument 54800 may have a power requirement (e.g., frequency, current, and voltage) that may be depend on the determined energy modality. Surgical instrument 54800 may determine an appropriate energy modality based on outer tube 54811. For example, surgical instrument 54800 may determine or select to deliver ultrasonic energy for a first dimension or type of outer tube 54811, and may determine or select to deliver RF energy for a second (e.g., different) type of waveguide (e.g., that may have a different dimension, shape, and/or configuration).

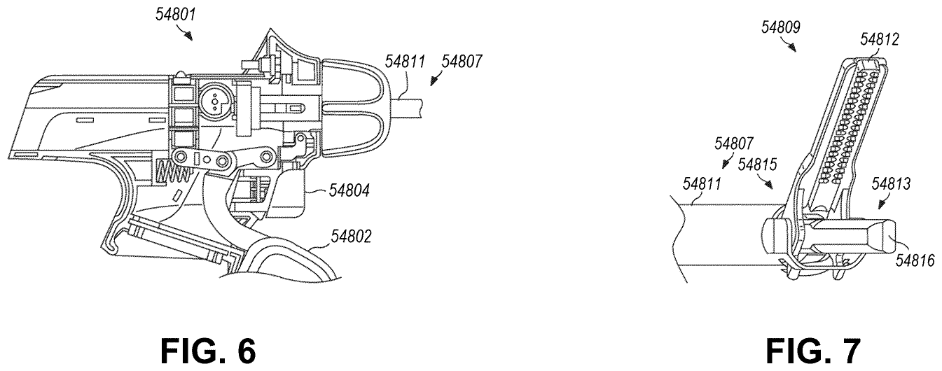

FIG. 6 depicts an example handle assembly 54801 of the surgical instrument 54800 shown in FIG. 5, including an example trigger 54802 and switch 54804. Trigger 54802 may be operably coupled to jaw member 54812 of end effector 54809. Blade 54813 may be configured to delivery ultrasonic energy or RF energy. Blade 54813 may be energized by transducer/generator assembly 54803 upon activating activation of switch 54804.

Trigger 54802 may activate end effector 54809, which may have a cooperative association with blade 54813 of waveguide shaft assembly 54807 (e.g., to enable various kinds of contact between end effector jaw member 54812 and blade 54813 with tissue and/or other substances). Jaw member 54812 of end effector 54809 may be a pivoting jaw. Jaw member 54812 may be used to grasp or clamp onto tissue disposed between jaw member 54812 and blade 54813. In examples, a feedback may be provided for one or more of the following: jaw member 54812 is engaged on tissue or other substances (e.g., by the trigger producing a click when it is fully depressed); an activation mode is determined or selected; an attachment is detected; a surgical task is detected; or energy cannot be delivered.

The feedback may be audible, visual, and/or mechanical (e.g., haptic) feedback. Audible feedback may be generated by a part (e.g., a thin metal part) that the trigger snaps over when it is depressed. Feedback may signal (e.g., inform a user) that the jaw is fully compressed against the waveguide 54816 and that sufficient clamping pressure may be being applied to accomplish vessel sealing. Feedback may be used to signal (e.g., inform a user) that a particular energy modality will be delivered. For example, a first audio cue may be used to represent when ultrasonic energy will be delivered by blade 54813 and a second audio cue may be used to represent when RF energy will be delivered by blade 54813.

Sensors, such as force sensors (e.g., strain gages or pressure sensors), may be coupled to trigger 54802 to measure the force applied to trigger 54802 (e.g., by a user). In examples, force sensors (e.g., strain gages or pressure sensors) may be coupled to switch 54804 such that displacement intensity corresponds to the force applied to switch 54804 (e.g., by a user). In examples, one or more sensors (e.g., a force sensor) may be used to determine or detect an actuation event (e.g., an actuation sequence of a button or switch of surgical instrument 54800).

Activation switch 54804 may be used to place surgical instrument 54800 into an operating mode (e.g., an ultrasonic operating mode or an RF operating mode). In an ultrasonic operating mode, depression of activation switch 54804 may cause ultrasonic motion at waveguide shaft assembly 54807. In an RF operating mode, depression of activation switch 54804 may cause RF energy to be delivered by waveguide shaft assembly 54807.

Depression of activation switch 54804 may cause electrical contacts within a switch to close, thereby completing a circuit between smart battery assembly 54805 and transducer/generator assembly 54803 (e.g., so that electrical power may be applied to the transducer). In examples, closing contacts, opening contacts, and/or processor-controlled power delivery may include receiving information from switch 54804 and may include directing a corresponding circuit reaction based on the information.

FIG. 7 depicts a fragmentary, enlarged perspective view of an example end effector 54809 of the surgical instrument 54800 shown in FIG. 5 (e.g., from a distal end with a jaw member 54812 in an open position). End effector 54809 may be used to collectively refer to jaw member 54812, the inner tube, outer tube 54811, and blade 54813 of waveguide 54816.

Referring to FIG. 7, waveguide shaft assembly 54807 may include an outer tube 54811 surrounding a portion of the waveguide. Blade 54813 of waveguide 54816 may protrude from distal end 54815 of outer tube 54811. Blade 54813 may contact the tissue during a medical procedure and may transfer energy (e.g., ultrasonic energy or RF energy) to the tissue. Waveguide shaft assembly 54807 may include jaw member 54812. Jaw member 54812 may be coupled to outer tube 54811 and an inner tube. Outer tube 54811 and the inner tube may slide longitudinally with respect to each other. As the relative movement between outer tube 54811 and the inner tube occurs, the jaw member 54812 may pivot (e.g., upon a pivot point) causing the jaw member 54812 to open and close. When closed, jaw member 54812 may impart a pinching force on tissue located between jaw member 54812 and the blade 54813 (e.g., ensuring positive and efficient blade-to-tissue contact).

Surgical instrument 54800 may monitor and/or collect usage data associated with the position and movement of surgical instrument 54800 and/or associated with user inputs, such as those relating to controlling jaws for clamping tissue. Healthcare professionals operating surgical instrument 54800 may be monitored using sensing systems (e.g., advanced imaging systems, such as environmental sensing system 20015) to collect data associated with movement of body parts, heartrate, respiration, temperature, and the like. The usage data and sensing data may be communicated to a surgical computing system (e.g., a generator or surgical hub, such as surgical hub 20006).

Surgical instrument 54800 may comprise or communicatively couple sensor(s) configured to provide electrical, mechanical, and/or visual data. Sensor(s) may include position sensors, strain gauges (e.g., micro-strain gauges), load sensors, proximity sensors, thermometers, gyroscopes, image sensors, and the like.

A position sensor may comprise any number of magnetic sensing elements, such as, for example, magnetic sensors (e.g., classified according to whether they measure the total magnetic field or the vector components of the magnetic field). The position sensor may be interfaced with the microcontroller 54818 to provide an absolute positioning system.

A strain gauge or a micro-strain gauge may be configured to measure one or more parameters of end effector 54809, such as the amplitude of the strain exerted on blade 54813 during a clamping operation. The amplitude of strain exerted on blade 54813 and/or displacement of blade 54813 during use may be indicative of closure forces applied to blade 54813 (e.g., jaw force, clamp force). The measured strain may be converted to a digital signal and provided to processor 54819.

A load sensor may be used to measure the force applied to the tissue by the end effector (e.g., jaw force). A load sensor may be coupled to the end effector (e.g., jaw member 54812 of surgical instrument 54800) to measure the force on the tissue being treated by the end effector. A load sensor for may be used to determine tissue characteristic(s) of a tissue grasped by the end effector. For example, a load force sensor may measure the amplitude and/or magnitude, which may be indicative of the tissue compression. A load sensor may measure the firing force applied to a closure member coupled to a clamp arm of the surgical instrument or the force applied by a clamp arm to tissue located in the jaws of the surgical instrument.

A current sensor may be configured to measure the current drawn by a motor of surgical instrument 54800. A magnetic field sensor may be employed to measure the thickness of the captured tissue. The measurement of the magnetic field sensor also may be converted to a digital signal and provided to the processor 54819.

Other sensor(s) may be provided to measure physical parameters of surgical instrument 54800 and/or a surgical site. In examples, the other sensor(s) can include sensor arrangements such as those described in U.S. Pat. No. 9,345,481 (U.S. patent application Ser. No. 13/800,025), titled STAPLE CARTRIDGE TISSUE THICKNESS SENSOR SYSTEM, filed on Mar. 13, 2013; U.S. Patent Application Publication No. 2014/0263552 (U.S. patent application Ser. No. 13/800,067), titled STAPLE CARTRIDGE TISSUE THICKNESS SENSOR SYSTEM, filed on Mar. 13, 2013; and U.S. Pat. No. 10,881,399 (U.S. patent application Ser. No. 15/628,175), titled TECHNIQUES FOR ADAPTIVE CONTROL OF MOTOR VELOCITY OF A SURGICAL STAPLING AND CUTTING INSTRUMENT, filed Jun. 20, 2017, which are herein incorporated by reference in their entirety.

Measurements of tissue compression, tissue thickness, and/or force required to close the end effector on a tissue, as respectively measured by the sensor(s) may be used by surgical instrument 54800 to characterize the selected position of the firing member and/or the corresponding value of the speed of the firing member. In examples, a memory 54820 may store a technique, an equation, and/or a lookup table which can be used by the microcontroller 54818 to determine a tissue characteristic.

Transducer/generator assembly 54803 may be modeled as an equivalent series resonant circuit. Transducer/generator assembly 54803 may comprise a first branch having a static capacitance and a second branch (e.g., a motional branch) having a serially connected inductance, resistance and capacitance that define the electromechanical properties of a resonator. Surgical instrument 54800 may have an initial electromechanical resonant frequency (e.g., defined by the physical properties of the transducer/generator assembly 54803, waveguide 54816, and/or ultrasonic blade 54813). Transducer/generator assembly 54803 may be excited by an alternating voltage Vg(t) and current Ig(t) signal at a frequency equal to the electromechanical resonant frequency (e.g., the resonant frequency of surgical instrument 54800). When surgical instrument 54800 is excited at the resonant frequency the phase angle φ between the voltage Vg(t) and current Ig(t) signals may be zero.

In examples, surgical instrument 54800 may be configured for ultrasonic activation modes (e.g., for transecting and/or coagulating tissue during surgical procedures). Surgical instrument 54800 may be configured for transecting, coagulating, scaling, welding and/or desiccating tissue during surgical procedures, for example.

Surgical instrument 54800 may use modular shafts that accomplish end effector functions. The energy modality may be selectable based on a measure of specific measured tissue and instrument characteristics, such as, for example, electrical impedance, tissue impedance, electric motor current, jaw gap, tissue thickness, tissue compression, tissue type, temperature, among other characteristics, or a combination thereof, to determine a suitable energy modality algorithm to employ ultrasonic vibration and/or electrosurgical high-frequency current to carry out surgical coagulation/cutting treatments on the living tissue based on the measured tissue characteristics identified by the surgical instrument. Once the tissue characteristics have been identified, the surgical instrument may be configured to control treatment energy applied to the tissue (e.g., in a single or segmented RF electrode configuration or in an ultrasonic configuration) through the measurement of specific tissue/device characteristics. Tissue treatment algorithms are described in commonly owned U.S. Pat. No. 11,141,213 (U.S. patent application Ser. No. 15/177,430) titled SURGICAL INSTRUMENT WITH USER ADAPTABLE TECHNIQUES, filed on Jun. 9, 2016, which is herein incorporated by reference in its entirety.

During a surgical procedure, a surgeon may have a reduced ability to focus on external factors, such as the status of surgical devices not under their control. Monitoring consistency of data and/or determining surgical site conditions, such as a proximity of surgical instrument 54800 to other surgical devices, tissue, and other surgeons or staff, may enable surgical instrument 54800 to proactively identify hazardous uses of surgical instrument 54800.

It may be difficult for a surgeon to adjust electrosurgical settings (e.g., select an activation mode) during a procedure due to the need for precise control and the risk of unintended tissue damage or injury to the patient. Surgeons may be concerned about injury to nearby biological structures (e.g., tissue, organs) either by direct contact or by the visually unrecognizable transmission of energy. Manually refining energy levels and selection of activation modes of an electrosurgical instrument may interfere (e.g., prolong and/or complicate) a surgical procedure. Real-time dynamic selection of activation mode(s) based on monitored data may alleviate these concerns and allow for finer adjustments to energy levels than otherwise practical if performed manually.

Surgeons should be familiar with the capabilities and limitations of electrosurgical instruments and their generators, as well as the specific techniques and procedures for using them (e.g., in different surgical specialties). It may be desirable to expand functionality without adding user interface complexity.

Reducing the user interface and/or frequency of required user input for surgical instrument 54800, for example by reducing the number of input sources (e.g., buttons, switches, interfaces, etc.), may minimize user interface complexity and streamline user experience (e.g., for new users). Delivery of an energy associated with an activation mode (e.g., energy modality) may be based on actuation of a single input source (e.g., a button) of the surgical instrument. For example, surgical instrument 54800 may comprise a single user input source (e.g., button) to trigger activation mode selection.

Dynamic determination of an activation mode of the surgical instrument based on monitored data may enable simplification of user interaction with the surgical instrument. Adaptive activation mode selection using a single input source may expand functionality of the surgical instrument while minimizing user interface complexity. Activation mode selection may be performed by surgical instrument 54800, at an energy generator, at a surgical hub, and/or the like.

A single actuation source (e.g., button) may be used to dynamically select an activation mode of surgical instrument 54800. The surgical instrument may be configured to operate in a plurality of activation modes that may be associated with different energy modalities. For example, the surgical instrument may be configured to deliver monopolar RF, bipolar RF, ultrasonic (e.g., tissue welding), and/or a combination of these energy modalities for combined therapeutic effect.

In examples, an optimal activation mode may be selected when the button is pressed based on monitored data (e.g., data monitored by a generator/surgical hub/surgical instrument 54800). For example electrical, mechanical, and/or visual data may be used to situationally determine an activation mode and/or to enable the device to enter multiple operating conditions at multiple modalities (e.g., RF, ultrasonic) using a single activation button.

Operating conditions may be determined and/or an activation mode may be selected based on monitored data. For example, at low operating temperatures RF energy may be useful for removing, shrinking, and/or sculpting soft tissue while simultaneously sealing blood vessels. RF energy may work well on connective tissue, which may be primarily comprised of collagen (e.g., which may shrink when contacted by heat). Low frequencies may be used for bipolar applications, for example if a risk analysis shows the possibility of neuromuscular stimulation has been mitigated to an acceptable level. Frequencies above 5 MHz may not be used (e.g., in order to minimize the problems associated with high frequency leakage currents). High frequencies may, however, be used in the case of bipolar applications (e.g., 10 mA may by a lower threshold of thermal effects on tissue). In examples, a single button may be used to deliver a sequence of combined ultrasonic and RF energy based on sensed data.

Systems, methods, and instrumentalities as disclosed herein for dynamically determining an activation mode of a surgical instrument (e.g., an electrosurgical instrument) based on monitored data may simplifying the user experience (e.g., during a surgical procedure) and minimize human error while achieving a wider range of system applicability.

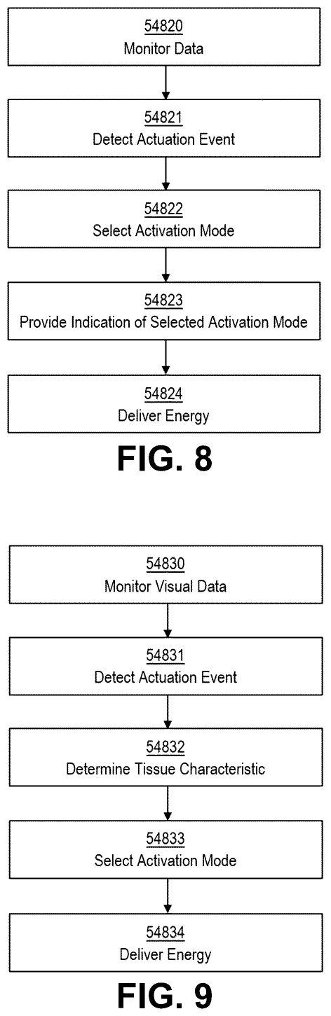

Dynamic determination of an activation mode of surgical instrument 54800 may be based on monitored data (e.g., visual data, electrical data, and/or mechanical data). FIG. 8 depicts an example method of dynamically selecting an activation mode of a surgical instrument (e.g., based on monitored data).

As illustrated in FIG. 8, at 54820, data may be monitored (e.g., by surgical instrument 54800 and/or a surgical system 20002 as described herein with respect to FIGS. 1-3). Monitored data may include one or more of: visual data, electrical data, or mechanical data. Monitoring data may include internally generated data stream(s) (e.g., collected by surgical instrument 54800 or the surgical system 20002 to which the surgical instrument may belong) and/or externally supplied data stream(s) (e.g., received from other surgical systems 20003, 20004 and/or sub-systems).

Visual data may be received from an advanced imaging system. Visual data may comprise at least one of: MRI data, ultra sound data, (EBUS) data, or camera data. Electrical data may indicate at least one of: an impedance, a temperature, or a force encountered by a jaw of the surgical instrument (e.g., a tissue characteristic encountered by the jaw). Mechanical data may indicate at least one of: a jaw gap position of the jaw, a clamp force of the jaw, a stapler closure load force, or a cartridge selection.

At 54821, an actuation event may be detected (e.g., by surgical instrument 54800). Actuation events may be based on one or more of: actuation force, a number of actuations, actuation duration, or time since a previous actuation. In examples, the actuation event may initiate a data retrieval mode (e.g., enhanced data monitoring, for example from an additional data source, and/or retrieval of up to date data) and/or a tissue analysis mode (e.g., trigger 54822, data interpretation). For example, the button actuation event may be used to transfer data feeds between two separate smart devices (e.g., which may enable the devices to synchronize their operations).

An actuation event may include temporal considerations. For example, if an activation mode hasn't been changed in X seconds (e.g., 2 seconds), surgical instrument 54800 may interpret a next actuation to be a distinct actuation event.

Surgical instrument 54800 may include a single input source, such as a button. An input source may be a multi-stage button or a single signal button. A multi-stage button may be a continuous signal button (e.g., using a hall effect) to determine separate (e.g., two) levels of button actuation. A multi-stage button may enable the system to re-configure the “threshold” ranges of the stages of actuation based on externally provided data streams (e.g., enabling detection of a varied set of actuation events). Multiple discrete levels (e.g., two discrete levels) of button actuation may be detected based on pressing in differing locations within the button and/or at differing force. Movement levels of the multi stage button may be used to indicate distinct user intentions.

A single signal button, which may be referred to as a multiple interpretation button, may be used. Monitored data (e.g., internal and/or external data stream(s) may be used to determine the operation of the button and the destination of the operation. The multiple interpretation outputs of the button may be used to transfer data feeds to two separate smart devices (e.g., which may enable the devices to synchronize their operations with respect to each other).

Surgical instrument 54800 may include a button memory, for example to record actuation history and/or interpret actuation events (e.g., sequences). The button memory may enable mode retainment and reset-ability, and/or more complex actuation events. For example, pressing the button multiple times and/or holding the button may be used to create a set of unique activation modes. For example, an actuation event may include three actuations with two quick actuations indicating an energy modality, such as an ultrasonic activation mode, and a duration of the third actuation indicating a particular ultrasonic activation mode (e.g., power level).

Surgical instrument 54800 may consider a time since last activation (e.g., include delays before switching activation modes). For example, surgical instrument 54800 may retain a current device activation mode if activation is occurring within every X seconds (e.g., 10 seconds). After X seconds has been passed, the retained current device activation mode may be reset (e.g., consider switching activation mode on next activation).

With continued reference to FIG. 8, at 54822 an activation mode may be selected. Activation modes may be (pre)configured operation parameters (e.g., for surgical instrument 54800). Activation modes may be associate with an energy modality and/or an energy level (e.g., power level). In examples, activation modes may be associated with an intended use, such as cutting or coagulation.

Monitored data may be used to inform activation mode selection. Monitored data may be used to determine one or more of: a tissue characteristic, an instrument characteristic, or a surgical procedure (e.g., a surgical task).

Surgical instrument 54800 may use monitored data to determine a tissue characteristic of a tissue within a surgical site. A tissue characteristic may be one or more of: a tissue thickness, an organ associated with the tissue, a tissue composition, a tissue location, a tissue tension, or a tissue deformation. Determined tissue characteristics may be used to select an activation mode. For example, if tissue impedance is determined to be below a threshold an activation mode associated with ultrasonic energy may be selected (e.g., to cut the tissue), and if tissue impedance is determined to be above a threshold an activation mode associated with RF energy may be selected (e.g., to seal the tissue).

Surgical instrument 54800 may use monitored data to determine an instrument characteristic (e.g., a characteristic of surgical instrument 54800). A instrument characteristic may be one or more of: a location of the surgical instrument within a surgical site (e.g., relative to a tissue), an orientation of the surgical instrument, displacement of blade 54813 of surgical instrument 54800, a position of jaw member 54812 of surgical instrument 54800, a force applied by jaw member 54812 of surgical instrument 54800, or a proximity to another surgical device. Determined instrument characteristics may be used to select an activation mode. For example, if monitored data indicates that the jaw of the surgical instrument is closed on a tissue an activation mode associated with RF energy may be selected.

Surgical instrument 54800 may use monitored data to determine a surgical task being performed. The surgical task may be part of a larger surgical procedure that may be identified using data of personnel characteristics (e.g., surgeon location, surgeon orientation), instrument characteristics, and/or tissue characteristics. For example, monitored data may be used to determined when a transection is complete to transition from an activation mode associated with coagulation to an activation mode associated with cutting. Monitored data may be used to determine progress through transection, such as by observing steam, blanching of tissue, change in tissue thickness, and/or heat (e.g., IR camera, or thermometer).

Surgical instrument 54800 may use surgical context information, for example in conjunction with the monitored data (e.g., visual, mechanical, electrical), to determine the activation mode. Surgical context information may include data associated with best practices, a clinician's preferences, and patient and/or procedure specific details. For example, the contextual information may include data related to patient biomarker baselines, surgical plan information, flaws in surgery data collection, administered agents, patient sensitivities, potential interactions, instruction for use, surgical procedure baselines, and the like. The surgical context may provide context for the surgical procedure and/or elements of a surgical procedure. For example, surgical context information may include data associated with a lung surgical procedure. The surgical context may be used to identify elements in a visual data (e.g., a surgical video), such as a lung. The surgical context may be used to distinguish elements in the visual data from other similar elements. For example, organs may share a similar shape in a surgical video, but with the surgical context, the proper organ may be identified.

Surgical context information may be obtained from a situationally aware surgical system 5100 as illustrated in FIG. 4. For example, data sources 5126 may provide parameters associated with patient monitoring devices 5124, HCP monitoring devices 35510, a surgical environment, and/or modular device 5102 (e.g., surgical instrument 54800). For example, the surgical hub 5104 may derive the contextual information pertaining to the surgical procedure from the data based upon, for example, the particular combination(s) of received data or the particular order in which the data is received from the data sources 5126. The contextual information as described with regard to FIG. 4 may be used to select an activation mode. The situational awareness of surgical instrument 54800 may be used to determine intermediate characteristics.

Surgical instrument 54800 may recognize and/or receive user input of a surgical procedure and may use stored or received data regarding the planned procedure to better identify surgical steps (e.g., as the surgery is performed). For example, a (pre)determined sequence of energy modalities may be delivered if a sequence of surgical steps of the procedure are known.

During data interpretation, monitored data, for example visual data, electrical data, and/or mechanical data, may be analyzed for consistency. Consideration and/or weighting (e.g., by the surgical instrument) of indications from monitored data may depend on a determined consistency of the data. For example, surgical instrument 54800 may determine whether an activation mode is consistent with monitored data across sources (e.g., prior to selection of the activation mode). If a determination is made that the activation mode is consistent (e.g., across types of data and/or data sources), the activation mode may be selected (e.g., selection of the activation mode is conditional on data consistency).

Although described with respect surgical instrument 54800, it should be appreciated that determination of an activation mode may be made remote to surgical instrument 54800 (e.g., by surgical hub 2006). For example, surgical instrument 54800 may analyze collected data (e.g., from sensor(s) of surgical instrument 54800) and data received from another smart device to enable a decision maker (e.g., surgical instrument 54800, surgical hub 20006, or other sub-system, for example a generator such as generator module 20050) to select an activation mode.

As shown in FIG. 8, at 54823, surgical instrument 54800 may provide a user (e.g., a surgeon) with an indication of a selected activation mode. The surgical instrument may indicate the selected activation mode via one or more of: an audible indication, a visual indication, or haptic feedback. For example, an LED of surgical instrument 54800 may light up with a color associated with the currently selected activation mode. In some examples, indicating the selected activation mode may be bypassed.

The surgical instrument may be configured to receive user feedback to confirm whether the user intends to use the selected activation mode (e.g., if an activation mode was selected). If a user feedback indication is a confirmation of the selected activation mode, the surgical instrument may deliver energy of the energy modality and/or energy level associated with the selected activation mode at 54825.

An indication of an activation mode currently selected (e.g., based on a detected actuation event and/or monitored data) may be provided to a user. An indication of activation mode selection may enable a user to overwrite the predictive mode selection. The indication may be visual (e.g., an LED, such as a multicolor LED), audible, or mechanical (e.g., using haptics). indicate which mode the surgical instrument is in. The indication may allow for a button press to cycle between activation modes (e.g., via a press and hold).

Mechanical indications may include haptic feedback, such as a continuous pulse to indicate to the user which operation mode will be initiated. Haptic feedback may be provided via the input source (e.g., button haptic feedback rather than haptic feedback across the device). Haptic feedback may be delivered using a vibration model that may, for example incorporate a magnet. A number of pulses and/or duration of pulses may be used to distinguish different modes.

Visual indications may include illumination pattern(s) and/or colors of an LED (e.g., a different color may signify a different mode). For example, an LED on a clamp arm of the surgical instrument that may be used to illuminate the jaws/tissue in the jaws of the surgical instrument may be configured to provide visual feedback (e.g., which may enable a clinician to maintain focus on a surgical site). In examples, a currently selected activation mode may be indicated on a monitor.

Audible indications may include a sequence of sounds and/or a pronunciation of the selected activation mode (e.g., “ultrasonic mode”). A first standard tone may be associated with ultrasonic energy and a second standard tone may be associated with RF energy (e.g., RF sealing). Audible indications may be provided by a generator and/or a device communicatively coupled with surgical instrument 54800.

As shown in FIG. 8, at 54824, an energy associated with the selected activation mode may be delivered by surgical instrument 54800 (e.g., via blade 54813).

Monitored data may be used to minimize inadvertent activation mode selection. For example, if determined impedance (e.g., at end effector 54809) is below a threshold that may indicate no tissue is disposed between jaw member 54812 and blade 54813 and that activation is inadvertent. For example, if an actuation event is brief (e.g., less than 1 second) that may indicate inadvertent activation. If inadvertent activation is determined to have occurred, no energy may be delivered.

Dynamic determination of an activation mode of surgical instrument 54800 may be based on monitored visual data. FIG. 9 depicts an example method of selecting an activation mode based on monitored visual data.

As shown in FIG. 9, at 54830 visual data may be monitored. Surgical instrument 54800 may be configured with communication, sensing, and/or feedback capabilities to monitor (e.g., receive and/or collect) visual data (e.g., visual data associated with a surgical procedure). Monitored data may include an internally generated data stream(s) and/or an externally supplied data stream(s) (e.g., data received from other surgical instrument(s), device(s), and/or system(s) to determine or select an appropriate activation mode. Externally supplied visual data streams may be received from, for example, surgical hubs and/or imaging systems (e.g., MRI, ultra sound, endobronchial ultrasound (EBUS), and/or camera, such as an IR camera). Surgical instrument 54800 may monitor visual data from multiple sources. For example, imaging systems may include environmental sensing system 20015 and/or imaging module 20049 as shown in FIG. 3, and/or patient monitoring devices 5124, HCO monitoring devices 35510, and/or environment monitoring devices 35512 as shown in FIG. 4.

Imaging systems may be used to allow surgical instrument 54800 to interpret a surgical site and/or one or more elements thereof, such as a patient's status. Monitored visual data may be local and/or remote to a surgical theater (e.g., surgical operating room 20035 as shown in FIG. 2). For example, visual data may include images and/or video taken before or during a surgical procedure (e.g., radiological patient images, such as pre-operative CT, intra-operative CT, and fluoroscopy). Imaging systems in a surgical setting may include scopes with cameras that view the surgical site and transmit the visual data to surgical instrument 54800.

Imaging systems may be limited by the information that may be recognized and/or conveyed. For example, certain concealed structures, physical contours, and/or dimensions within a three-dimensional space (e.g., of a patient's tissue) may be unrecognizable intraoperatively by certain imaging systems. Additionally, certain imaging systems may be incapable of processing certain information to the clinician(s) intraoperatively. Surgical instrument 54800 may overcome these challenges by analyzing visual data from multiple sources to provide a more wholistic view of the surgical site and/or verify accuracy/consistency of determined characteristics.

As shown in FIG. 9, at 54831 an actuation event (e.g., a sequence of one or more button presses) may be detected by surgical instrument 54800. The actuation event may include one or more of: an actuation force, a number of button actuations, and/or an actuation duration (e.g., as described herein).

At 54832 monitored visual data may be used to determine a tissue characteristic of a tissue in a surgical site (e.g., in response to the actuation event at 54831). The tissue characteristic may include one or more of a tissue thickness, an organ associated with the tissue, a tissue composition, a tissue location, a tissue tension, and/or a tissue deformation.

At 54833 surgical instrument 54800 may determine an activation mode based on monitored visual data. For example, the activation mode may be determined based on one or more of tissue characteristics, instrument characteristics, or clinician characteristics, which may be derived from monitored visual data.

At 54834 an energy associated with the selected activation mode may be delivered by surgical instrument 54800 (e.g., via blade 54813).

A selected activation mode may be updated as visual data is monitored. For example, a selected activation mode and/or a predicted activation mode may change as visual data and/or determined intermediate characteristics change. For example, a tissue characteristic determined based on visual data at a first time (e.g., associated with a first actuation event) may be different than a tissue characteristic determined based on visual data at a second time (e.g., associated with a second actuation event).

A tissue characteristic (e.g., each tissue characteristic) may be associated with one or more activation modes. An association between a tissue characteristic and an activation mode may be positive (e.g., if the tissue characteristic is determined then the activation mode may be chosen) or negative (e.g., if the tissue characteristic is determined then the activation mode should be disabled or a warning should be provided to a clinician before delivering energy).

A composition of a tissue (e.g., a tissue type) may be determined by surgical instrument 54800 based on monitored visual data. The tissue composition may include, for example, the ratio of collagen to elastin in the tissue, the stiffness of the tissue, and/or the thickness of the tissue. For example, visual data of a tissue grasped by or at the end effector 54809 of surgical instrument 54800 may be analyzed to determine if the tissue is composed of collagen base material or connective material.

A thickness of a tissue may be determined by surgical instrument 54800 based on monitored visual data. Tissue thickness may affect the operation of surgical instrument 54800 and/or the success of a surgical procedure. For example, tissue thickness may be important in determining the risk of a post-operative leak.

Surgical instrument 54800 may adjust the activation mode (e.g., a selected energy modality) throughout the course of the surgical procedure (e.g., rather than just on a procedure-by-procedure basis), for example based on a tissue characteristic, such as tissue thickness. For example, when working with thick tissue surgical instrument 54800 may start in a coagulation activation mode (e.g., RF coagulate). Surgical instrument 54800 may interrogate an impedance curve to determine when the activation mode should be adjusted to a cut mode (e.g., RF cut).

Tissue tension of a tissue may be determined by surgical instrument 54800 based on monitored visual data. Visual analysis of strain measurements, tissue coloration, and tissue deformation may be performed by surgical instrument 54800. For example, a deformed vessel may be a visual indication of tissue tension. Tissue tension may be determined and adjusted throughout a sealing cycle to ensure minimal tension.

Determined tissue tension may be compared against a threshold, for example to determine whether an action, such as temporarily disabling an activation mode, should be taken. For example, if determined tissue tension surpasses the threshold, a notification may be provided to a clinician and/or activation modes associated with RF energy may be temporarily disabled (e.g., as applying RF energy may not be appropriate during tissue tension). In examples, a first activation mode (e.g., associated with RF energy) may be selected if a determined tissue tension is below a threshold value, and the second activation mode (e.g., an activation mode associated with ultrasonic energy) may be selected if the tissue tension is above the threshold value.

A location of a tissue may be determined by surgical instrument 54800 based on monitored visual data. Tissue location may be used for organ identification. For example, if visual data provided by an imaging system indicates surgical instrument 54800 is to be used on the liver, the activation mode associated with ultrasonic energy may be selected (e.g., to support hemostasis needs).

Tissue location may be used to assess the tissue and a condition of a surrounding area. For example, skeletonization of a vein may be detected before surgical instrument 54800 is clamped on the vein. Surgical instrument 54800 may determine a hemostasis activation mode should be activated (e.g., ultrasonic energy should be delivered) based on the skeletonization (e.g., situational awareness).

Surgical instrument 54800 may determine, based on the visualization data, an instrument characteristic (e.g., of surgical instrument 54800 and/or other surgical instrument(s)/device(s)). The instrument characteristic may include a location of the surgical instrument within the surgical site, an orientation of the surgical instrument, a current surgical task, and/or its proximity to a second surgical instrument. Surgical instrument 54800 may select an activation mode based on the instrument characteristic. Surgical instrument 54800 may select an activation mode based on determined instrument characteristics of other surgical devices. For example, if surgical instrument 54800 determines it is in close proximity to a second surgical instrument (e.g., based on visual data), surgical instrument 54800 may temporarily disable an activation mode and/or provide a warning to a clinician.

Monitored visual data may be used to determine an instrument characteristic of surgical instrument 54800 or a surgical device. Instrument characteristics may include a blade location, a jaw location, a device identity, orientation of a device (e.g., relative to a tissue), and/or a user of a device. Surgical instrument 54800 may select an activation mode based on one or more instrument characteristics. An instrument characteristic (e.g., each instrument characteristic) may be associated with one or more activation modes.

Monitored visual data may be used to identify/determine a current surgical task and/or surgical procedure. Visual data in a surgical setting may be used to determine clinician characteristics. A clinician characteristic may be associated with a healthcare professional's visual focus, task being performed, position, and/or visual signal (e.g., a gesture associated with a command). Monitoring imaging systems located in the operating room may track the motions of a clinician, which may include tracking eye motions and/or locations, to determine the focus of attention of the clinician. Surgical instrument 54800 may determine a clinician is located at a particular angle or location relative to the surgical site that is associated with a particular surgical task. A surgical task and/or surgical procedure may be associated with one or more activation modes.

Visual data may be used to determine relative progress of a surgical task. For example, surgical instrument 54800 may determine when a transection is complete and may transition from a coagulation activation mode to a cut activation mode by observing steam, blanching of tissue, change in thickness, or heat (e.g., using an IR camera).

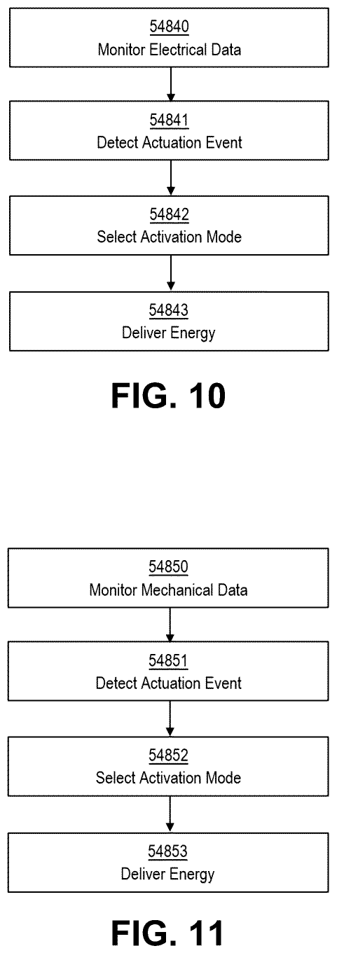

Dynamic determination of an activation mode of surgical instrument 54800 may be based on monitored electrical data. FIG. 10 depicts an example method of selecting an activation mode based on monitored electrical data.

As shown in FIG. 10, at 54840, electrical data may be monitored. Surgical instrument 54800 may be configured with communication, sensing, and/or feedback capabilities to monitor (e.g., receive and/or collect) electrical data (e.g., electrical data associated with a surgical procedure). Monitored data may include an internally generated data stream(s) and/or an externally supplied data stream(s) (e.g., data received from other surgical instrument(s), device(s), and/or system(s) described herein) to determine or select an appropriate activation mode. Externally supplied electrical data streams may be received from, for example, surgical hubs and/or surgical devices (e.g., a surgical stapling device). Surgical instrument 54800 may monitor electrical data from multiple sources. For example, a first electrical data source may indicate a stapler has just been used, and a second electrical data source may indicate tissue impedance, capacitance, and/or device pressure on tissue (e.g., as collected/determined by surgical instrument 54800).

As shown in FIG. 10, at 54841, an actuation event (e.g., a sequence of one or more button presses) may be detected by surgical instrument 54800. The actuation event may include one or more of: an actuation force, a number of button actuations, and/or an actuation duration (e.g., as described herein).

At 54842, surgical instrument 54800 may determine an activation mode based on the monitored electrical data. For example, the activation mode may be selected based on one or more of tissue characteristics, instrument characteristics, or surgical tasks, which may be derived from monitored electrical data.

At 54843, an energy associated with the selected activation mode may be delivered by surgical instrument 54800 (e.g., via blade 54813).

Local sensors of surgical instrument 54800 (e.g., force sensor, position sensor) may be used to determine a current state/status of surgical instrument. The sensed data may be used to interpret a surgical site (e.g., one or more elements thereof, such as a patient's status). For example, electrical data may be used to determine a tissue characteristic of a tissue in a surgical site (e.g., in response to the actuation event at 54831). The tissue characteristic may include one or more of, for example, an impedance of the tissue, a density of the tissue, a temperature of the tissue, or a resistance (e.g., force against surgical instrument) of the tissue.

One or more tissue characteristics may be associated with one or more activation modes. An association between a tissue characteristic and an activation mode may be positive (e.g., if the tissue characteristic is determined then the activation mode may be chosen) or negative (e.g., if the tissue characteristic is determined then the activation mode should be disabled or a warning should be provided to a clinician before delivering energy).

Surgical instrument 54800 may determine, based on the electrical data, an impedance and/or a capacitance associated with a tissue in the surgical site. Surgical instrument 54800 (e.g., in conjunction with a generator, such as generator module 20050 as shown in FIG. 3) may determine tissue impedance, for example via a control loop for measuring impedance and/or capacitance of tissue. For example, electrical continuity across a jaw equipped with electrodes may be employed to assess a tissue (e.g., determine what percentage of the jaw is covered with the tissue). In examples, tissue characteristics may be determined from data collected following a sub-therapeutic pulse.

A composition of a tissue (e.g., a tissue type) may be determined by surgical instrument 54800 based on monitored electrical data. The tissue composition may include, for example, the ratio of collagen to elastin in the tissue, the stiffness of the tissue, and/or the thickness of the tissue. For example electrical data of a tissue grasped by or at the end effector 54809 of surgical instrument 54800 may be analyzed to determine if the tissue is composed of collagen base material or connective material (e.g., based on a sub-therapeutic pulse). Density ranges or patterns (e.g., (pre)defined mappings of tissue impedance) may be used to determine a composition of tissue.

Tissue density may be detected/determined based on impedance, temperature, and/or force encountered by end effector 54809 of surgical instrument 54800. For example, the impedance of tissue encountered by end effector may vary based on the density of contacted tissue. For instance, a relatively dense tissue (e.g., scar tissue) may exhibit relatively high impedance as compared to impedance exhibited by a less dense tissue (e.g., fat tissue). Sensor(s) (e.g., sensors of surgical device 54800 or a separate surgical device) may be used to detect changes in tissue density as a function of impedance. Impedance may include electrical impedance and/or acoustic impedance. For instance, dense tissue may exhibit both high electrical impedance and high acoustic impedance.

Tissue characteristics may be determined using dynamic mechanical analysis (DMA), which may also be referred to as mechanical spectroscopy. DMA may be a technique used to study and/or characterize materials by applying a sinusoidal stress applied to the material. A strain in the material may be measured, enabling a determination of a complex modulus of the material. DMA may be applied to ultrasonic devices by exciting the tip of the ultrasonic blade with a sweep of frequencies (e.g., compound signals or frequency sweeps) and measuring the resulting complex impedance at each frequency.

Surgical device 54800 may determine an activation mode based on a determined tissue characteristic. For example, if tissue impedance is determined to be below a first threshold, an ultrasonic activation mode may be selected (e.g., to cut the tissue), whereas if the tissue impedance is above a second threshold, a high-frequency current activation mode (e.g., associated with RF energy) may be selected (e.g., to seal the tissue).

Some examples of determining and employing tissue characteristics may be found in U.S. Pat. No. 10,765,470 (U.S. patent application Ser. No. 15/177,466), titled SURGICAL SYSTEM WITH USER ADAPTABLE TECHNIQUES EMPLOYING SIMULTANEOUS ENERGY MODALITIES BASED ON TISSUE PARAMETERS, filed December Jun. 9, 2016, the disclosure of which is incorporated herein by reference in its entirety.

Monitored electrical data may be used to determine instrument characteristics, for example of surgical instrument 54800 and/or other surgical devices. An instrument characteristic may include a location, an orientation, available activation modes, a currently selected activation mode, a time of last use, a duration of use, a charge, and the like. For example, firing of a surgical stapling device in the same surgical site as surgical instrument 54800 may inform activation mode selection. For example, if a surgical stapler was recently used, an activation mode associated with ultrasonic energy may be selected (e.g., to support hemostasis needs), and activation mode(s) associated with RF energy may be disabled (e.g., as RF energy may be dangerous when used in close proximity to staples). If a clinician wants to use a disabled activation mode (e.g., in this instance RF energy), the clinician may be required to acknowledge a warning/hazard. Indications (e.g., warnings, prompts, alerts) may be provided by surgical instrument 54800 to a user if unsafe or potentially hazardous site conditions are determined.

Monitored electrical data may be used to identify/determine a current surgical task and/or surgical procedure (e.g., based on tissue characteristics, instrument characteristics, and/contextual data). A surgical task and/or surgical procedure may be associated with one or more activation modes.

A sequence of activation modes (e.g., energy modalities) may be selected based on a (pre)configured procedure. For example, three actuation events during a surgical procedure may require an ultrasonic activation mode and a fourth (e.g., sequentially) may require an RF activation mode. A surgical procedure or desired sequence of activation modes may be specified (e.g., by a clinician), for example prior to beginning surgery.

Surgical instrument 54800 may determine an activation mode based on temporal indications (e.g., time since a previous actuation) from electrical data (e.g., internally recorded/monitored). For example, surgical instrument 54800 may include delays in between switching activation modes. For example, surgical instrument 54800 may retain an activation mode if a button actuation event is occurring at a particular frequency (e.g., every 3 seconds). For example, surgical instrument 54800 may have a default activation mode that is automatically selected after a delay (e.g., a window of inactivity, such as 5 minutes).

Electrical data may include reporting data and/or feedback (e.g., from a clinician). Feedback may be used to confirm a surgical task (e.g., transection) is underway or has been completed. Actions taken (e.g., by surgical instrument 54800 or a surgical device) may be reported to surgical device 54800 or a surgical hub. Recording actions and/or indications (e.g., warnings) issued during a surgical procedure may be stored (e.g., for later verification). Surgical instrument 54800 and/or a communicatively coupled surgical system (e.g., surgical hub 20006 as shown in FIG. 1) may record data pertaining to a surgical procedure for generating reports, verifying the steps being taken by the surgeon to perform the surgical procedure, and/or providing data or prompts that may be pertinent for a particular procedural step.

Dynamic determination of an activation mode of surgical instrument 54800 may be based on monitored mechanical data. FIG. 11 depicts an example method of selecting an activation mode based on monitored mechanical data.

As shown in FIG. 11, at 54850 mechanical data may be monitored. Surgical instrument 54800 may be configured with communication, sensing, and/or feedback capabilities to monitor (e.g., receive and/or collect) mechanical data (e.g., mechanical data associated with a surgical procedure). Monitored data may include an internally generated data stream(s) and/or an externally supplied data stream(s) (e.g., data received from other surgical instrument(s), device(s), and/or system(s) to determine or select an appropriate activation mode. Surgical instrument 54800 may monitor mechanical data from multiple sources.

Sensor(s) (e.g., sensors of surgical instrument 54800) may be used to allow surgical instrument 54800 to interpret instrument operating conditions, for example when in use at a surgical site. For example, sensor(s) of surgical device 54800 and/or other surgical devices (e.g., an intelligent instrument 20014 as shown in FIG. 1) may detect various instrument characteristics. Instrument characteristics may include one or more of: connection characteristics (e.g., wired and/or wireless connection to a surgical hub, loading unit, generator, or power source); jaw characteristics (e.g., jaw gap position, jaw force); blade deflection; drive shaft characteristics (e.g., rotation, torque, strain); temperature; cartridge selection; or firing characteristics (e.g., a number of firings, a peak force during firing, a total amount of force, a number of pauses during firing, etc.).

Monitored mechanical data may be associated with surgical instrument 54800. The mechanical data may include a jaw gap position indication (e.g., fully open, partially closed, fully closed) associated with a jaw of the surgical instrument; a tissue presence indication (e.g., associated with a jaw of the surgical instrument), and/or a jaw force indication (e.g., a detected jaw force, detected load). The surgical instrument may select an activation mode based on the jaw gap position indication, the tissue presence indication, and/or the jaw force indication.

Mechanical data may be used to determine instrument characteristic(s) associated with device functions (e.g., dissect, clamp, coagulate, cut, staple, etc.) of an end effector assembly. Mechanical data of surgical instrument 54800 may be obtained from, for example, one or more of a jaw gap sensor, a jaw force sensor, or an emitter (e.g., to determine blade deflection). A jaw gap position sensor may be used to determine a degree of jaw positioning of jaw member 54812 (e.g., to determine whether a jaw is fully open, partially open, or closed). A jaw force sensor may be used to determine force required to open/close jaw member 54812. An emitter may be used to determined blade deflection of blade 54813 (e.g., a distance between blade 54813 and an inner tube of surgical instrument 54800). Monitored mechanical data may be used by surgical instrument 54800 to select an activation mode and/or to determine tissue characteristics. For example, if a jaw gap is determined to be larger than a 5 mm gap, an ultrasonic activation mode may be selected.

Jaw position may be determined based on monitored tissue contact data and/or signals from sensor(s) of surgical instrument 54800. Surgical instrument 54800 may determine an initial point of contact between end effector 54809 and a tissue being clamped. A position of the jaws at the initial tissue contact point may be determined by detecting when at least one of the sensors disposed on each of the jaws detects tissue contact. Surgical instrument 54800 may determine the position of the jaws according to a sensed distance or gap between the sensor(s), a sensed position of the closure tube, or a sensed angle at which the jaw(s) are oriented.

Determined jaw position may be evaluated based on (e.g. in conjunction with) with detected actuation event(s). A jaw gap position (e.g., whether jaws are fully open, partially open, or closed) may be determined at a time of actuation (e.g., to inform activation mode selection). For example, if jaw member 54812 is determined to be in a closed position and a tissue is present, an RF activation mode may be selected, and if jaw member 54812 is determined to be in a closed position and no tissue is present, an ultrasonic activation mode may be selected.

Surgical instrument 54800 may determine an activation mode based on temporal indications (e.g., time since a previous actuation) from mechanical data (e.g., internally recorded/monitored). For example, surgical instrument 54800 may consider one duration of jaw position (e.g., whether the jaw member 54812 has maintained a jaw state, for example closed, for a period of time). Surgical instrument 54800 may retain an energy modality associated with one or more activation mode(s) if a button actuation event is occurring at a particular frequency (e.g., every 3 seconds). For example, if repeated small incremental motion is determined and an ultrasonic activation mode is selected, surgical instrument 54800 may continue to deliver ultrasonic energy. In such examples, the power of a selected ultrasonic activation mode may be increased (e.g., as this repetitive behavior may be indicative of tissue marching and/or tip bite marching). Surgical instrument 54800 may have a default activation mode that is automatically selected after a delay (e.g., a window of inactivity, such as 5 minutes).

A jaw force (e.g., a force required to open or close jaw member 54812) may be determined by surgical instrument 54800 (e.g., to inform activation mode selection).

Surgical instrument 54800 may determine jaw force by using sensor(s) positioned at the jaws of end effector 54809. The sensor(s) may be a strain gauge coupled to end effector 54809, which may be configured to measure the magnitude/amplitude of strain on jaw member 54812 of the end effector, which may be indicative of closure forces being applied. The sensor may be a load sensor configured to measure a closure force applied to jaw member 54812 and/or blade 54813 by a closure drive system. In examples, a current sensor may be used to measure a current drawn by the motor, which may correlate to a closure force applied to jaw member 54812 and/or blade 54813.

Tissue characteristics may be determined by surgical instrument 54800 based on monitored mechanical data (e.g., obtained jaw position indication(s) and/or jaw force indication(s)). A force sensor may measure the force and torque generated by a motor. The motor may be configured to actuate end effector 54809 of surgical instrument 54800, for example to realize clamping, rotation, and/or articulation functionality. A motor controller may determine tissue characteristics of clamped tissue (e.g., density of tissue).

Surgical instrument 54800 may determine a load, such as a closure force applied by end effector 54809. Load may be measured via a load sensor of surgical instrument 54800. For example, if jaws of surgical instrument 54800 are closed from a fully open state and a clamp load is observed that is below a threshold, an ultrasonic activation mode may be selected (e.g., for feathering).

A clamp force may be determined by surgical instrument 54800 based on blade deflection. Blade deflection may be measured by an imaging system (e.g., an external imaging system with a view of surgical instrument 54800) and/or an emitter measuring the distance between blade 54813 and an inner tube/clevis. If a measurement indicates the blade is not centered (e.g., deflection is observed) it may be determined that something, such as a tissue, is caused blade to deflect and an ultrasonic activation mode associated may be selected (e.g., only ultrasonic activation modes may be enabled).

Blade deflection may be used to ensure safe operating conditions for surgical instrument 54800. For example, if blade deflection is detected to surpass a threshold, a user may be prompted to reposition surgical instrument 54800 and/or run a device assessment to determine usability under the detected blade deflection condition.

Force applied on outer tube 54811 of surgical instrument 54800 (e.g., from a trocar) may be monitored. A high force on the outer tube may indicate tension is being applied to tissue and/or that the an ultrasonic activation mode should be selected (e.g., as tissue tension may not be optimal for RF). For example, surgical instrument 54800 may determine an ultrasonic activation mode should be selected if a force is being applied to outer tube 54811, jaw member 54812 is open, and an actuation event is detected.

As shown in FIG. 11, at 54851, an actuation event (e.g., a sequence of one or more button presses) may be detected by surgical instrument 54800. The actuation event may include one or more of: an actuation force, a number of button actuations, and/or an actuation duration (e.g., as described herein).

At 54852, surgical instrument 54800 may determine an activation mode based on the monitored electrical data. For example, the activation mode may be determined based on one or more of tissue characteristics, instrument characteristics, or surgical tasks, which may be derived, at least in part, based on the monitored electrical data.

Surgical instrument 54800 may determine, based on mechanical data, a progression of surgical tasks in a surgical procedure and may coordinate activation mode selection based on such progression. For example, surgical instrument 54800 may determine the surgical tasks associated with the surgical procedure plan and may select an activation mode that is associated with a current surgical task.

At 54853, an energy associated with the selected activation mode may be delivered by surgical instrument 54800 (e.g., via blade 54813).

During a surgical procedure, multiple sources of data may need to be managed and/or leveraged to improve patient outcomes without increasing complexity for clinicians. Data sources may provide (e.g., may each provide) distinct types of data. Aggregating data and/or verifying consistency of data may be necessary to effectively select an activation mode for surgical instrument 54800. For example, determined intermediate results (e.g., tissue characteristics, instrument characteristics, surgical tasks) may be based on multiple data sources.

It may be difficult for a surgeon to adjust electrosurgical settings (e.g., select an activation mode) during a surgical procedure based on available data associated with the surgical procedure. Surgeons may be concerned about injury to nearby biological structures (e.g., tissue, organs), status of the patient, locations and orientations of other devices, and the like, but may be unable to effectively consider the data associated with these considerations. Manually refining energy levels and selection of activation modes of an electrosurgical instrument may interfere (e.g., prolong and/or complicate) a surgical procedure. Real-time dynamic selection of activation mode(s) based on monitored data may alleviate these concerns and allow for finer adjustments to energy levels than otherwise practical if performed manually.

Monitored data sources (e.g., sources of visual, electrical, and/or mechanical data) may be aggregated and/or parsed to situationally determine an activation mode. Monitored data may be compared for consistency, precision, and/or accuracy when determining intermediate characteristic(s) (e.g., tissue characteristics, instrument characteristics, current surgical task) that may be used during adaptive activation mode selection. Data retrieval and/or analysis modes may be initiated based on use of the surgical instrument (e.g., to efficiently poll for data, compare data at particular time instances, etc.).

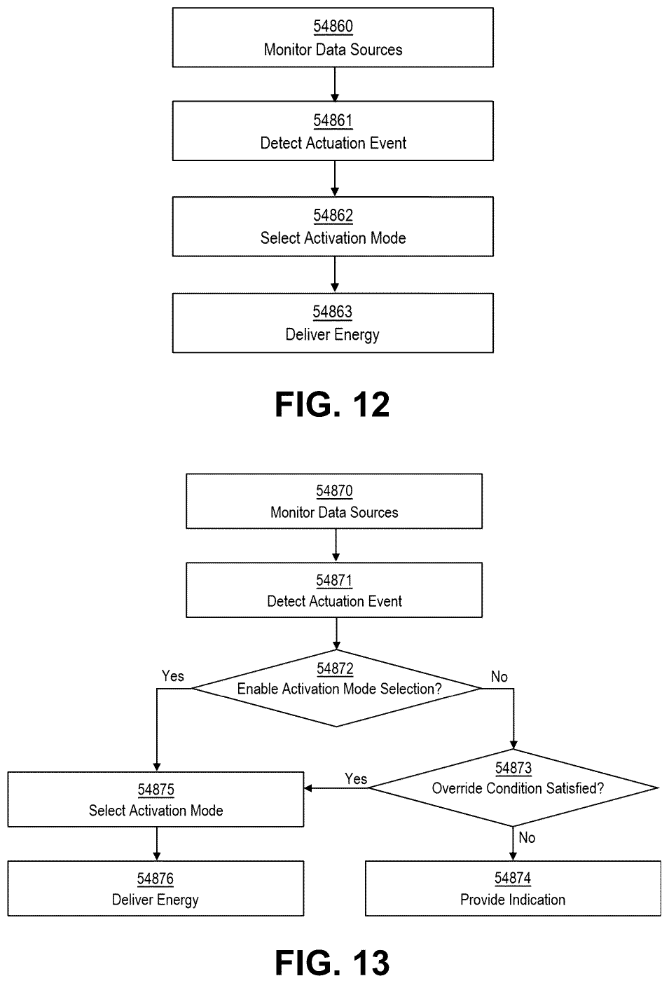

Dynamic determination of an activation mode of surgical instrument 54800 may be based on multiple sources of monitored data (e.g., visual data, electrical data, and/or mechanical data). FIG. 12 depicts an example method of dynamically selecting an activation mode of a surgical instrument (e.g., based on monitored data).

As illustrated in FIG. 12, at 54860, data may be monitored (e.g., by surgical instrument 54800 and/or a surgical system 20002. Monitored data may include one or more of: visual data, electrical data, or mechanical data. Monitoring data may comprise internally generated data stream(s) (e.g., collected by surgical instrument 54800 or the surgical system 20002 to which the surgical instrument may belong) and/or externally supplied data stream(s) (e.g., received from other surgical systems 20003, 20004 and/or sub-systems). External data stream(s) may be obtained (e.g., directly obtained) from other devices or systems (e.g. an imagining system, a surgical stapler, a smoke evacuator, etc.), such as via a wired or wireless connection. Surgical instrument 54800 may have a wired connection (e.g., be plugged into) a surgical hub/generator, so that data may be exchanged via a wired, high speed, and more reliable connection. In examples, monitored data may be continuously or periodically obtained.

Visual data may be received from an advanced imaging system. Visual data may comprise at least one of: MRI data, ultra sound data, (EBUS) data, or camera data. Electrical data may indicate at least one of: an impedance, a temperature, or a force encountered by a jaw of the surgical instrument (e.g., a tissue characteristic encountered by the jaw). Mechanical data may indicate at least one of: a jaw gap position of the jaw, a clamp force of the jaw, a stapler closure load force, or a cartridge selection.

Surgical instrument 54800 may be configured to use a data retrieval and analysis mode to obtain monitored data. A data retrieval and analysis mode may be triggered when an actuation event is detected and/or when surgical instrument 54800 senses movement or repositioning and anticipates upcoming use. For example, when actuation force (e.g., any actuation force, such as more than 0 lbs of force and/or more than 1 mm of button displacement) is detected, surgical instrument 54800 may enter data retrieval mode and obtain (e.g., request) data from all systems and devices that are capable of communicating with it. In examples, some data source(s) may be constantly monitored and a data retrieval analysis mode may include retrieving data from additional data source(s).

A data recovery process may be triggered upon an ongoing data transfer (e.g., from a monitored data source) being interrupted/broken. During a data recovery process, surgical instrument 54800 may check other data sources (e.g., surrounding devices) for missing data. For example, surgical instrument 54800 may check visual data from an imaging system to determine a location of a device that had an interruption in data transmission. Multiple data sources may be necessitated by one data source being broken. A data recovery process may trigger a data retrieval analysis mode and/or retrieving data from a additional data source(s).

Surgical instrument 54800 may include an internal gyroscope and/or movement sensing sensor. A data retrieval and analysis mode may be initiated, for example when device movement is observed and after device movement has been stagnant.

At 54861, an actuation event may be detected (e.g., by surgical instrument 54800). Actuation events may be based on one or more of: actuation force, a number of actuations, actuation duration, or time since a previous actuation. In examples, the actuation event may initiate a data retrieval mode (e.g., enhanced data monitoring, for example from an additional data source, and/or retrieval of up-to-date data) and/or a tissue analysis mode (e.g., trigger 54822, data interpretation). For example, the button actuation event may be used to transfer data feeds between two separate smart devices (e.g., which may enable the devices to synchronize their operations).

At 54862 an activation mode may be selected. Activation modes may be (pre)configured operation parameters (e.g., for surgical instrument 54800). Activation modes may be associate with an energy modality and/or an energy level (e.g., power level). In examples, activation modes may be associated with an intended use, such as cutting or coagulation.

An activation mode may be associated with multiple conditions (e.g., the conditions may be required for activation, or any of the conditions may be sufficient for activation). Conditions may be based on multiple data sources and/or a determined consistency of the data sources. A indication (e.g., notification, confirmation) may be provided to a user if the conditions for activating a particular activation mode are not all met.

In examples, a combination of internally generated data stream(s) and an externally supplied data stream(s) may be used to resolve indecisive indicators. For example, if condition A is observed from visual data, and electrical data from the surgical instrument confirms an anticipated tissue electrical feedback response, the surgical instrument may use an activation mode based on condition A. For example, mechanical force may be used to differentiate between two visual indicators (e.g., visual data sources) that are indecisive.

Intermediate characteristic(s) (e.g., tissue characteristics, instrument characteristics, current surgical task) that may be used during adaptive activation mode selection may be determined for a data source (e.g., each data source) or a type of data (e.g., each type of data, for example visual, electrical, and/or mechanical data). Determined intermediate characteristics(s) may be compared, for example for consistency and/or appropriateness.

For example, if a tissue is determined to have a tissue characteristic (e.g., high tissue density) based on a first data type (e.g., visual data), a second data source (e.g., electrical data) may be used to confirm (e.g., improve confidence in) the determined tissue characteristic. If a first data source and a second data source lead to conflicting intermediate characteristic(s), the determined tissue characteristic(s) may not be used in activation mode selection and/or may be weighted appropriately when selecting an activation mode.

Surgical instrument 54800 may select an activation mode based on intermediate characteristics from different data source(s) and/or data type(s).

For example, mechanical data may indicate a sequence of rapid jaw closures of a jaw of the surgical instrument and visual data may be used to determine a tissue thickness of a tissue within the jaw. Based on these intermediate characteristics, surgical instrument 54800 may compare the the tissue thickness to a thickness threshold, for example to identify the surgical task of feathering. If the tissue thickness is above the thickness threshold surgical instrument 54800 may determine feathering is being performed and/or select an ultrasonic activation mode (e.g., maintain an ultrasonic activation mode for the duration of feathering).

For example, mechanical data may indicate a jaw force of a jaw of the surgical instrument and visual data may indicate whether blade deflection of a blade of the surgical instrument has occurred. If blade deflection has occurred, an ultrasonic activation mode may be selected.

For example, mechanical data may indicate a jaw of the surgical instrument is closed and electrical data may be used to determine an impedance of a tissue location in the jaw. If the tissue impedance is above a threshold impedance, a RF activation mode may be selected.

Activation mode(s) may be predicted for a data source (e.g., each data source) or a type of data (e.g., each type of data, for example visual, electrical, and/or mechanical data). Predicted activation mode(s) may be compared, for example for consistency and/or appropriateness. For example, if predicted activation modes are inconsistent across data sources, activation mode selection may be disabled.

Although described with respect surgical instrument 54800, it should be appreciated that determination of an activation mode may be made remote to surgical instrument 54800 (e.g., by surgical hub 2006). For example, surgical instrument 54800 may analyze collected data (e.g., from sensor(s) of surgical instrument 54800) and data received from another smart device to enable a decision maker (e.g., surgical instrument 54800, surgical hub 20006, or other sub-system, for example a generator such as generator module 20050) to select an activation mode.

As shown in FIG. 12, at 54863, an energy associated with the selected activation mode may be delivered by surgical instrument 54800 (e.g., via blade 54813).

In examples, surgical instrument 54800 may provide an indication of the selected activation mode, wherein the indication is at least one of: a visual indication, an audible indication, or haptic feedback. User feedback (e.g., confirmation of the indication of the selected activation mode) may be required prior to energy delivery. User feedback may include a desired activation mode (e.g., a manually determined activation mode).

Dynamic determination of an activation mode of surgical instrument 54800 may be based on multiple sources of monitored data (e.g., visual data, electrical data, and/or mechanical data). FIG. 13 depicts an example method of dynamically selecting an activation mode of a surgical instrument (e.g., based on monitored data).

As illustrated in FIG. 13, at 54870, data may be monitored (e.g., by surgical instrument 54800 and/or a surgical system 20002. As described herein, monitored data may include one or more of: visual data, electrical data, or mechanical data. At 54871, an actuation event may be detected (e.g., by surgical instrument 54800). Actuation events may be based on one or more of: actuation force, a number of actuations, actuation duration, or time since a previous actuation. In examples, the actuation event may initiate a data retrieval mode (e.g., enhanced data monitoring, for example from an additional data source, and/or retrieval of up to date data) and/or a tissue analysis mode (e.g., trigger 54822, data interpretation). For example, the button actuation event may be used to transfer data feeds between two separate smart devices (e.g., which may enable the devices to synchronize their operations).

As illustrated in FIG. 13, at 54872 a determination of whether to enable activation mode selection may be made. Activation mode selection may be not be performed (e.g., be disabled) based on one or more of: data incompleteness (e.g., interruption of monitored data); data inconsistencies (e.g., between determined intermediate characteristics and/or activation modes based on data from different sources); a determined hazard (e.g., location of the surgical instrument relative to biological structures or other surgical devices); or user preference/feedback. An indication of whether activation mode selection is performed may be provided to a user, for example via mechanical, visual, and/or audible indicators. The indication may include one or more reasons(s) for or preventing activation mode selection (e.g., incompatibilities).

Surgical instrument may be configured to operate in a continuous activation mode. A continuous activation mode may enable clinicians to continually activate a particular activation mode (e.g. using a single source). In a continuous activation mode, activation mode selection may not be performed. For example, a continuous activation mode may be selected when the jaws of the surgical instrument are considered open by sub therapeutic pulse (e.g., the surgical instrument may stop looking for open circuits).

Activation mode selection may be prevented based on data inconsistencies or incompleteness, and an indication (e.g., that adaptive activation mode selection is disabled and/or that no activation mode has been selected) may be provided to a user.

For example, if visual data (e.g., from an imaging system) indicates a tissue is present in the jaws of the surgical instrument but measured impedance indicates metal or non-tissue is in the jaws, no mode may be activated and/or a notification may be provided for a user to check/verify tissue. In such an example, RF activation modes may be disabled and/or ultrasonic activation modes may be used if a user satisfies an override condition (e.g., as illustrated in FIG. 13, at 54873).

For example, if visual data (e.g., from an imaging system) indicates transection is taking place in the lung, but electrical data indicates the tissue impedance of intestines, activation mode selection may be disabled until an override condition is satisfied by a user (e.g., as illustrated in FIG. 13, at 54873).

Intermediate characteristic(s) (e.g., tissue characteristics, instrument characteristics, current surgical task) that may be used during adaptive activation mode selection may be determined for a data source (e.g., each data source) or a type of data (e.g., each type of data, for example visual, electrical, and/or mechanical data). Determined intermediate characteristics(s) may be compared, for example for consistency and/or appropriateness. For example, if a tissue is determined to have a high tissue density (e.g., a tissue characteristic) based on a first data source and a low tissue density based on a second data source, the first data source and the second data source may be conflicting and neither determined tissue characteristic may be used for activation mode selection.

In examples, priority may be given to data source(s) and or type(s) for resolving conflicting intermediate characteristics. For example, if a first data source is more likely to accurately predict an intermediate characteristic than a second data source, the first data source may be considered (e.g., weighted) more than the second data source when determining the intermediate characteristic and/or during activation mode selection.

In examples, when activation mode selection is prevented (e.g., if there is a gap in data or data inconsistences), manual mode selection may be requested/required.

Adaptive activation mode selection may be prevented due to data corruption and/or data being outside of an expected range (e.g., to optimize patient treatment and minimize risk). Surgical instrument 54800 may disable particular activation modes or power levels, for example during conflict resolution due to data being unavailable, corrupted, or outside of expected range.

Adaptive activation mode selection may be prevented based on location and/or orientation of surgical instrument 54800 relative to biological structures and/or other surgical devices. In examples, limited adaptive activation mode selection may be permitted, for example with certain modes or power levels may be disabled from being selected due to incompatibilities. These safety precautions may be taken as surrounding devices may interfere with energy delivery (e.g., RF energy delivery) and/or nearby biological structures may be critical. For example, ultrasonic activation mode(s) may be disabled when surgical instrument 54800 is determined to be in close proximity to the ureter. For example, a power level of a selected activation mode may be reduced based on a proximity to a surgical device.

Particular activation modes may be disabled based on device alarms and/or errors. For example, if an RF error code occurs prohibiting future RF activation, an ultrasonic activation mode may be used when open jaw back cutting.

Particular activation modes may be disabled based on determined attachments and/or sensed attributes of surgical instrument 54800.

As illustrated in FIG. 13, at 54873 a clinician may satisfy an override condition to reenable adaptive activation mode selection and/or manually select an activation mode. In examples, an override condition may be an actuation event (e.g., a single button press) to confirm the clinician wants to use a particular activation mode (e.g., an activation mode that may be disabled).

At 54874, An indication may be provided that adaptive activation mode selection was not performed.

At 54875 an activation mode may be selected (e.g., if not disabled at 54872). Activation modes may be (pre)configured operation parameters (e.g., for surgical instrument 54800). Activation modes may be associate with an energy modality and/or an energy level (e.g., power level). In examples, activation modes may be associated with an intended use, such as cutting or coagulation.

An activation mode may be associated with multiple conditions (e.g., all conditions may be required for activation, or any of the conditions may be sufficient for activation). Conditions may be based on multiple data sources and/or a determined consistency of the data sources. A indication (e.g., notification, confirmation) may be provided to a user if the conditions for activating a particular activation mode are not all met.

In examples, a combination of internally generated data stream(s) and an externally supplied data stream(s) may be used to resolve indecisive indicators. For example, if condition A is observed from visual data and electrical data from the surgical instrument confirms an anticipated tissue electrical feedback response, the surgical instrument may use an activation mode based on condition A. For example, mechanical force may be used to differentiate between two visual indicators (e.g., visual data sources) that are indecisive.

Although described with respect surgical instrument 54800, it should be appreciated that determination of an activation mode may be made remote to surgical instrument 54800 (e.g., by surgical hub 2006). For example, surgical instrument 54800 may analyze collected data (e.g., from sensor(s) of surgical instrument 54800) and data received from another smart device to enable a decision maker (e.g., surgical instrument 54800, surgical hub 20006, or other sub-system, for example a generator such as generator module 20050) to select an activation mode.

As shown in FIG. 13, at 54876, an energy associated with the selected activation mode may be delivered by surgical instrument 54800 (e.g., via blade 54813).

What is claimed is:

1. A method for a surgical instrument configured to operate in a first activation mode associated with radio frequency (RF) energy and a second activation mode associated with ultrasonic energy, the method comprising:
monitoring first data from a first data source and second data from a second data source;
detecting an actuation event of a button of the surgical instrument;
determining, in response to the actuation event, whether to enable activation mode selection based on the first data and the second data;
selecting, in response to a determination to enable activation mode selection, an activation mode from the first activation mode and the second activation mode, wherein the selected activation mode is based on the first data and second data; and
delivering, in response to the determination to enable activation mode selection, an energy of an energy output modality associated with the selected activation mode.

2. The method of claim 1, further comprising:
providing, in response to a determination to disable activation mode selection, an indication that an activation mode is not selected.

3. The method of claim 1, wherein determining whether to enable activation mode selection further comprises:
determining a first predicted activation mode from the first activation mode and the second activation mode based on the first data; and
determining a second predicated activation mode from the first activation mode and the second activation mode based on the second data, wherein:
upon the first predicted activation mode and the second predicated activation mode being consistent, activation mode selection is enabled, and
upon the first predicted activation mode and the second predicated activation mode being inconsistent, activation mode selection is disabled.

4. The method of claim 1, wherein determining whether to enable activation mode selection further comprises:
determining whether the first data and the second data are consistent, wherein:
upon a determination that the first data and the second data are consistent, activation mode selection is enabled, and
upon a determination that the first data and the second data are inconsistent, activation mode selection is disabled.

5. The method of claim 1, wherein determining whether to enable activation mode selection further comprises:
determining a first tissue characteristic of a tissue in a surgical site based on the first data; and
determining a second tissue characteristic of the tissue based on the second data, wherein:
upon the first tissue characteristic and the second tissue characteristic being consistent, activation mode selection is enabled, and
upon the first tissue characteristic and the second tissue characteristic being inconsistent, activation mode selection is disabled.

6. The method of claim 1, wherein determining whether to enable activation mode selection further comprises:
detecting an interruption in monitoring the first data from the first data source, wherein activation mode selection is disabled based on the interruption.

7. The method of claim 1, further comprising:
determining, based on the first data and the second data, a proximity of the surgical instrument to a biological structure within a surgical site, wherein a power level of the energy of the energy output modality associated with the selected activation mode is based on the proximity of the surgical instrument to the biological structure.

8. The method of claim 1, further comprising:
determining, based on the first data and the second data, a proximity of the surgical instrument to a surgical device, wherein a power level of the energy of the energy output modality associated with the selected activation mode is based on the proximity of the surgical instrument to the surgical device.

9. The method of claim 1, further comprising:
providing an indication of the selected activation mode, wherein the indication is at least one of: a visual indication, an audible indication, or haptic feedback; and
receiving user feedback, wherein the user feedback indicates a confirmation of the selected activation mode, wherein delivering an energy of an energy output modality associated with the selected activation mode is based on the user feedback.

10. The method of claim 1, further comprising:
providing an indication of the selected activation mode, wherein the indication is at least one of: a visual indication, an audible indication, or haptic feedback;
receiving user feedback, wherein the user feedback indicates disapproval of the selected activation mode and a desired activation mode, wherein the energy of the energy output modality associated with the selected activation mode is not delivered based on the user feedback; and
delivering, in response to the user feedback, an energy of an energy output modality associated with the desired activation mode.

11. A surgical instrument configured to operate in a first activation mode associated with radio frequency (RF) energy and a second activation mode associated with ultrasonic energy, the surgical instrument comprising:
a processor configured to:
monitor first data from a first data source and second data from a second data source, wherein the first data and the second data are each at least one of electrical data, mechanical data, or visual data;
detect an actuation event of a button of the surgical instrument;
determine, in response to the actuation event, whether to enable activation mode selection based on the first data and the second data; and
select, based on the first data and the second data, an activation mode from the first activation mode and the second activation mode.

12. The surgical instrument of claim 11, wherein determining whether to enable activation mode selection further comprises:
determining whether the first data and the second data are consistent, wherein:
upon a determination that the first data and the second data are consistent, activation mode selection is enabled, and
upon a determination that the first data and the second data are inconsistent, activation mode selection is disabled.

13. The surgical instrument of claim 11, wherein determining whether to enable activation mode selection further comprises:
determining whether the first data and the second data are complete, wherein:
upon a determination that the first data and the second data are complete, activation mode selection is enabled, and
upon a determination that the first data and the second data are incomplete, activation mode selection is disabled.

14. The surgical instrument of claim 11, wherein the processor is further configured to:
deliver, in response to the determination to enable activation mode selection, an energy of an energy output modality associated with the selected activation mode.

15. The surgical instrument of claim 11, wherein the processor is further configured to:
provide, in response to a determination to disable activation mode selection, an indication that an activation mode is not selected; and
provide, in response to a determination to enable activation mode selection, an indication of the selected activation mode.
